(19) United States
(12) Patent Application Publication
(10) Pub. No.: US 2026/0092323 A1
(43) Pub. Date: Apr. 2, 2026

(54) MODIFIED NUCLEOTIDES WITH PEPTIDE SPACERS

(71) Applicant: 10x Genomics, Inc., Pleasanton, CA (US)

(72) Inventors: Justin COSTA, Union City, CA (US); Jeremy LACKEY, Foster City, CA (US); Zhuoran MA, Palo Alto, CA (US); Meiliana TJANDRA, Dublin, CA (US)

(21) Appl. No.: 19/344,118

(22) Filed: Sep. 29, 2025

Related U.S. Application Data
(60) Provisional application No. 63/701,447, filed on Sep. 30, 2024.

(51) Int. Cl. C12Q 1/6869; C07K 19/00; C12Q 1/48; C12Q 1/6876

(52) U.S. Cl. CPC C12Q 1/6869; C07K 19/00; C12Q 1/48; C12Q 1/6876; C12Y 207/07

(57) ABSTRACT

Methods, systems, and kits for sequencing a template nucleic acid molecule using a modified nucleotide molecule including a peptide spacer are provided. In some aspects, the modified nucleotide molecules are used in a sequencing reaction, such as an in situ sequencing reaction.

### CROSS-REFERENCE TO RELATED APPLICATIONS

This application claims the priority benefit of U.S. Provisional Application No. 63/701,447, filed on Sep. 30, 2024, entitled “MODIFIED NUCLEOTIDES WITH PEPTIDE SPACERS” the contents of which is incorporated herein in its entirety.

### REFERENCE TO AN ELECTRONIC SEQUENCE LISTING

The present application is being filed along with a Sequence Listing in electronic format. The Sequence Listing is provided as a file entitled 202412023600SeqList.xml, created Sep. 29, 2025, which is 4,829 bytes in size. The information in the electronic format of the Sequence Listing is incorporated by reference in its entirety.

### FIELD

The present disclosure relates in some aspects to peptide-modified nucleotide molecules and methods of sequencing using the peptide-modified nucleotide molecules.

### BACKGROUND

Nucleic acid sequencing is a versatile tool that helps scientists advance the understanding of biology and has wide-ranging applications in various fields, such as medical diagnostics, biotechnology, forensic biology, and virology. For example, in situ sequencing is an advanced sequencing method that provides spatial resolution of gene expression within preserved spatial architecture of a biological sample. A problem with in situ sequencing is that the sequencing reagents involved, such as detectable labels for detecting a nucleotide, can non-specifically bind to tissue. For example, hydrophilicity, pi stacking, and electrostatic interaction of fluorescent dye molecules with biomolecules present in the tissue can contribute to non-specific binding. Solutions are needed for improving the compatibility of sequencing reagents with tissue for in situ nucleic acid-based assays such as in situ sequencing.

### SUMMARY

In some aspects, provided herein is a peptide-modified nucleotide having the structure: N-L-P-D, wherein N is a nucleotide comprising a 3′ blocked sugar and a base, L is a linker, P is a peptide comprising a charged residue, and D is a dye, and wherein the linker is attached to the base of the nucleotide. In some embodiments, the 3′ blocked sugar comprises a ribose. In some embodiments, the 3′ blocked sugar comprises a 2′-deoxyribose. In some embodiments, a 5′ phosphoryl group of the sugar comprises a monophosphate, diphosphate, or triphosphate, optionally wherein the 5′ phosphoryl group is a triphosphate. In some embodiments, the 3′ blocked sugar comprises a 3′-OR. In some embodiments, the R is selected from the group consisting of: azidomethyl, allyl, methyl, methyl carbamate, hydroxymethyl, amine, ester, disulfide, sulfate and phosphate.

In some embodiments, the base is selected from the group consisting of: an adenine, an analogue of adenine, a cytosine, an analogue of cytosine, a guanine, an analogue of guanine, a thymine, an analogue of thymine, a uracil, and an analogue of uracil.

In some embodiments, the base is selected from the group consisting of: an adenine, an analogue of adenine, a cytosine, an analogue of cytosine, a guanine, an analogue of guanine, a thymine, and an analogue of thymine.

In some embodiments, the peptide is 4 to 30 amino acids in length.

In some embodiments, the more than 50% of residues of the peptide are charged residues.

In some embodiments, the peptide comprises a negatively charged residue. In some embodiments, the negatively charged residue comprises aspartic acid.

In some embodiments, the peptide comprises a positively charged residue. In some embodiments, the peptide comprises a negatively charged residue and a positively charged residue.

In some embodiments, the peptide comprises a cysteine residue or a cysteine derivative. In some embodiments, a sulfur of the cysteine derivative is attached to the dye or the linker. In some embodiments, a sulfur of the cysteine derivative is attached to the dye.

In some embodiments, the peptide comprises an amine attached to the dye or the linker. In some embodiments, the peptide comprises an amine attached to the linker. In some embodiments, the amine attached to the linker is a side chain amine or an N-terminal amine.

In some embodiments, the peptide comprises the sequence X1-(X2)ₙ-X3, wherein n is 3 to 50, X1 is any amino acid or derivative thereof and is attached to one of the dye or the linker, X2 is any amino acid, and X3 is lysine or a cysteine derivative attached to the other of the dye or the linker. In some embodiments, X1 is any amino acid or derivative thereof and is attached to the dye, X2 is any amino acid, and X3 is lysine or a cysteine derivative attached to the linker. In some embodiments, X1 is any amino acid or derivative thereof and is attached to the linker or the linker, X2 is any amino acid, and X3 is lysine or a cysteine derivative attached to the dye. In some embodiments, the peptide comprises the sequence X1-(X2)ₙ-X3, wherein n is 3 to 50, X1 is any amino acid or derivative thereof, X2 is any amino acid, and X3 is lysine or a cysteine derivative.

In some embodiments, the X1 is a lysine and comprises an N-terminally blocked amino group.

In some embodiments, the peptide has the structure X1-(X2)ₙ-X3, wherein: n is 3 to 50, X1 or X3 is a cysteine derivative comprising a sulfur attached to the linker, and: (a) X1 is the cysteine derivative, X3 or an instance of X2 is a lysine having an amino side chain attached to the dye moiety, or (b) when X3 is the cysteine derivative, either: (i) X1 is any amino acid having an N-terminus attached to the dye moiety, or (ii) X1 or an instance of X2 is a lysine having an amino acid chain attached to the dye moiety. In some embodiments, X3 is the cysteine derivative and X1 or an instance of X2 is the lysine having an amino acid chain attached to the dye moiety

The peptide-modified nucleotide of claim 23, wherein X1 comprises an N-terminally blocked amino group.

In some embodiments, the peptide comprises an enzymatically cleavable sequence. In some embodiments, the peptide comprises a protease cleavage amino acid sequence. In some embodiments, the enzymatically cleavable sequence comprises a DDDDKX (SEQ ID NO: 4), wherein X is Q or G. In some embodiments, the enzymatically cleavable sequence comprises ENLYFQG (SEQ ID NO: 3).

In some embodiments, the peptide comprises a cysteine residue, and wherein the linker comprises a sulfur forming a disulfide bond with the cysteine.

In some embodiments, the linker comprises a cleavable moiety. In some embodiments, the cleavable moiety is selected from the group consisting of: O-azido, disulfide, nitrobenzyl, phosphoryl, and ester.

In some embodiments, the linker comprises polyethylene glycol.

In some embodiments, the base of the nucleotide is a purine, and the linker is attached to the C5 position of the purine. In some embodiments, the base of the nucleotide is a pyrimidine, and the linker is attached to the C7 position of the pyrimidine.

In some embodiments, the dye is a fluorescent dye. In some embodiments, the fluorescent dye is selected from the group consisting of: coumarin dye or coumarin-derivative dye, cyanine dye or a cyanine-derivative dye, fluorescein dye or a fluorescein-derivative dye, rhodamine dye or a rhodamine-derivative dye, or phenoxazine dye or a phenoxazine-derivative dye.

In some aspects, provided herein is a composition comprising a plurality of peptide-modified nucleotides, comprising: first peptide-modified nucleotides as provided herein, having a first base type; and a second peptide-modified nucleotides as provided herein, having a second base type. In some embodiments, the dye of the first peptide-modified nucleotide molecules is a first dye, and the dye of the second peptide-modified nucleotide molecules is a second dye that differs from the first dye. In some embodiments, the first base type and the second base type are selected from any two of: (i) an adenine or an analogue of adenine, (ii) a cytosine or an analogue of cytosine, (iii) a guanine or an analogue of guanine, and (iv) a thymine or an analogue of thymine or a uracil or an analogue of uracil.

In some embodiments, the composition further includes third peptide-modified nucleotides as provided herein, having a third base type. In some embodiments, the dye of the third peptide-modified nucleotide molecules is a third dye that differs from the first dye and the second dye. In some embodiments, the first base type, the second base type, and the third base type are selected from any three of: (i) an adenine or an analogue of adenine, (ii) a cytosine or an analogue of cytosine, (iii) a guanine or an analogue of guanine, and (iv) a thymine or an analogue of thymine or a uracil or an analogue of uracil.

In some embodiments, the composition further comprises fourth nucleotide molecules having a fourth base type. In some embodiments, the fourth nucleotide molecules are not attached to a dye. In some embodiments, the fourth nucleotide molecules comprise fourth peptide-modified nucleotide molecules of any one of claims 1 to 36 attached to a fourth dye that differs from the first dye, the second dye, and the third dye. In some embodiments, the first base type, the second base type, the third base type and the fourth base type, respectively, comprise: (i) an adenine or an analogue of adenine, (ii) a cytosine or an analogue of cytosine, (iii) a guanine or an analogue of guanine, and (iv) a thymine or an analogue of thymine or a uracil or an analogue of uracil.

In some aspects, provided herein is a method for sequencing a template nucleic acid molecule comprising: (a) a first contacting step of contacting a priming strand bound to a template nucleic acid molecule with a polymerase and a first plurality of nucleotides comprising a first peptide-modified nucleotide as provided herein, thereby incorporating the peptide-modified nucleotide into a 3′ terminus of the priming strand; and (b) a first detecting step to detect a presence of the first peptide-modified nucleotide in the priming strand to identify a complementary nucleotide in the template nucleic acid molecule.

In some aspects, provided herein is a method for sequencing a template nucleic acid molecule comprising: (a) contacting a priming strand bound to a template nucleic acid molecule with a polymerase and a first plurality of nucleotides comprising a first peptide-modified nucleotide having the structure N-L-P-D, wherein: N is a nucleotide molecule comprising a 3′ blocked sugar and a base, L is a linker, P is a peptide, and D is a dye, thereby incorporating the peptide-modified nucleotide into a 3′ terminus of the priming strand; and (b) detecting a presence of the first peptide-modified nucleotide in the priming strand to identify a complementary nucleotide in the template nucleic acid molecule.

In some embodiments, the method further comprises, after the first detecting step, a step of removing the 3′ blocking group of the first peptide-modified nucleotide from the priming strand.

In some embodiments, the method further includes a second contacting step of contacting the priming strand with a polymerase and a second plurality of nucleotides comprising a second peptide-modified nucleotide as provided herein, thereby incorporating the second peptide-modified nucleotide into the priming strand, and a second detecting step to detect a presence of the second peptide-modified nucleotide in the priming strand to identify a complementary nucleotide in the template nucleic acid molecule.

In some embodiments, the method further includes, after detecting the presence of the second peptide-modified nucleotide, a step of removing the 3′ blocking group of the second peptide-modified nucleotide from the priming strand.

In some embodiments, the method further includes repeating a cycle of the previously described steps, for at least one additional cycle, thereby identifying an additional complementary nucleotide in the template nucleic acid molecule. In some embodiments, the method includes repeating the cycle for at least 2, 5, 10, 20, or 30 additional cycles.

In some embodiments, the linker comprises a cleavable moiety, and wherein the method further comprises, after the detecting, cleaving the cleavable linker to remove the dye.

In some embodiments, the cleavable moiety is photocleavable and the cleaving comprises exposing the peptide-modified nucleotide to UV light.

In some embodiments, the cleavable moiety is disulfide or an O-azido methyl, and the cleaving comprises contacting the peptide-modified nucleotide with a reducing agent.

In some embodiments, the peptide-modified nucleotide comprises an enzymatically cleavable sequence, and the method further comprises, after the detecting, enzymatically cleaving the enzymatically cleavable sequence, thereby releasing the dye.

In some embodiments, the peptide of the peptide-modified nucleotide has a carboxy-terminus (C-terminus), and the method further comprises, after the detecting, contacting the peptide-modified nucleotide with a carboxypeptidase, thereby releasing the dye.

In some embodiments, the template nucleic acid molecule comprises DNA. In some embodiments, the template nucleic acid molecule comprises RNA, optionally wherein the template nucleic acid molecule is an mRNA molecule.

In some embodiments, the template nucleic acid molecule comprises a target analyte nucleic acid molecule. In some embodiments, the template nucleic acid molecule comprises a barcode sequence associated with a target analyte.

In some embodiments, the method further includes, prior to the first contacting step, hybridizing a circularizable probe or probe set to the target analyte or to a labeling agent bound to the target analyte and ligating the circularizable probe or probe set to form a circularized probe, wherein the method further comprises performing rolling circle amplification of the circularized probe to generate the template nucleic acid molecule. In some embodiments, the circularizable probe or probe set is a padlock probe.

In some embodiments, the template nucleic acid molecule to be sequenced is attached to a solid support. In some embodiments, the template nucleic acid molecule is sequenced in situ in a cell sample or tissue sample. In some embodiments, the cell sample or tissue sample is attached to a solid support. In some embodiments, the cell sample comprises a layer of cells deposited on a surface.

In some aspects, provided herein is a kit for sequencing a template nucleic acid molecule comprising: (a) a plurality of peptide-modified nucleotides as described herein or a composition peptide-modified nucleotides as described herein, and (b) a polymerase.

In some embodiments, the kit further includes a primer designed to hybridize to a template nucleic acid molecule. In some embodiments, the kit further includes one or more additional reagents for in situ sequencing of a target analyte in a cell or tissue sample.

### DETAILED DESCRIPTION

Provided herein are peptide-modified (“P-modified”) nucleotide molecules and related compositions and methods of making and using P-modified nucleotide molecules. The P-modified nucleotide molecules may be used for nucleic acid-based assays in tissue, such as in situ sequencing. In certain embodiments, P-modified nucleotide molecules provide reduced tissue fouling and/or enhanced tissue compatibility as compared to dye-labeled nucleotide molecules lacking a peptide modification. In some embodiments, the peptide includes charged amino acid residues, and the charged peptide-modified nucleotide molecule has reduced tissue fouling as compared to a modified nucleotide molecule attached to a same dye molecule but lacking the peptide including charged residues.

I. Modified Nucleotide Molecules with a Peptide Spacer

The present disclosure provides peptide-modified (“P-modified”) nucleotide molecules. In some aspects, the peptide of the P-modified nucleotide molecule linked to a dye provides surprising benefits when performing an nucleic acid based assay in a cell or tissue sample. For example, using the P-modified nucleotide molecule may result in a decrease of autofluorescence, tissue fouling, and/or dye background as compared to using a nucleotide molecule that lacks a peptide spacer between the nucleotide molecule and the dye molecule. In some aspects, a P-modified nucleotide molecule is linked to a detectable label via a peptide spacer. In some aspects, the peptide spacer and the nucleotide molecule are linked via a linker molecule. In some aspects, the peptide spacer and the nucleotide molecule are not linked via a sugar-phosphate backbone linkage.

In some embodiments, the P-modified nucleotide molecule includes the structure: N-P-D, wherein N is a nucleotide comprising a sugar and a base, P is a peptide, and D is a detectable label, wherein the peptide is directly or indirectly connected to the base of the nucleotide.

In some embodiments, the P-modified nucleotide molecule has the structure: N-L-P-D, wherein N is a nucleotide, L is a linker, P is a peptide, and D is a detectable label. In some embodiments, the detectable label is a dye. In some embodiments, the linker is connected to the base of the nucleotide.

### Peptide Spacers

Peptide-modified nucleotide molecules as described herein include a peptide molecule. A peptide molecule includes a chain of two or more amino acid residues. Peptides typically include amino acid residues linked by peptide bonds. Amino acids of the peptide may be in the L-configuration or the D-configuration. In particular embodiments, the peptide comprises amino acids in the L-configuration. In some embodiments, the peptide comprises amino acids selected from the group consisting of: arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and tryptophan.

In some embodiments, the peptide comprising a charged residue. In some embodiments, the charged residue is selected from lysine, arginine, aspartic acid, and glutamic acid. In some embodiments, the charged residue is selected from lysine, arginine, aspartic acid, glutamic acid, and histidine. Histidine has a pKA of 6.4, and a small fraction of histidine molecules are positively charged in a solution at pH 7. In some embodiments, the peptide comprises a negatively charged residue. Negatively charged residues have a negative charge at pH 7 and include aspartic acid and glutamic acid. In some embodiments, the peptide comprises a positively charged residue. Positively charged residues have a positive charge at pH 7 and include lysine and arginine.

In some embodiments, the peptide includes one or more negatively charged amino acids. Aspartic acid and glutamic acid are negatively charged amino acids. In particular embodiments, at least 30%, at least 40%, at least 50%, at least 60%, at least 70%, or at least 80% of the amino acid residues of the peptide are negatively charged. In particular embodiments, the peptide is 4-40 amino acid residues in length, and at least 30%, at least 40%, at least 50%, at least 60%, at least 70%, or at least 80% of the amino acid residues of the peptide are negatively charged. In particular embodiments, the peptide includes at least one glutamic acid. In particular embodiments, the peptide includes at least one aspartic acid. In some embodiments, the peptide includes four to twenty negatively charged residues. In some embodiments, the peptide includes six negatively charged residues.

In particular embodiments, the peptide includes at least one cysteine residue or a cysteine derivative. In some embodiments, the amino acid at the C-terminus of the peptide is cysteine. In some embodiments, the amino acid at the N-terminus of the peptide is cysteine. A cysteine derivative may be generated by coupling an N-terminal or C-terminal cysteine with an attachment moiety. A cysteine of a peptide may be modified using an electron deficient alkene/alkyne, for example, by a Michael addition with maleimide. As another example, NHS esters can be converted into thioesters, which can then react with proteins that have an N-terminal cysteine to form a new amide bond. In some embodiments, a sulfur of the cysteine is attached to the linker (e.g., via a disulfide linkage). In some embodiments, a sulfur of the cysteine is attached to the dye (e.g., via a disulfide linkage).

In some embodiments, an amine of the peptide is attached to the linker or the dye. In some embodiments, an amine is attached to the linker. In some embodiments, the amine is attached to the dye. The amine may be a side chain amine or an N-terminal amine. In some embodiments, an N-terminal amine is attached to the linker or the dye. In some embodiments, the peptide comprises a lysine residue. In some embodiments, the amino acid at the C-terminus of the peptide is lysine. In some embodiments, the amino acid at the N-terminus of the peptide is lysine. In some embodiments, a lysine of the peptide is conjugated to an attachment moiety (e.g., to the dye or a linker attaching to the nucleotide molecule). Examples of reagents that may generate an attachment moiety to a lysine include NHS esters sulfonyl fluorides, iminoboronates, diazonium salts, aldehydes, isothiocyanates.

In some embodiments, the peptide comprises a cysteine residue. In particular embodiments, the sulfur of the cysteine residue forms a disulfide bond with an attachment moiety. In particular embodiments, the cysteine forms a disulfide bond with an attachment moiety that links the peptide to the nucleotide molecule (e.g., via a linker moiety).

In some embodiments, an N-terminal amine of the peptide is not used as an attachment moiety for both the dye and the linker, and the N-terminal amine is blocked. In some embodiments, the N-terminal blocking group is acetylated.

In some embodiments, the peptide comprises the sequence X1-(X2)ₙ-X3, wherein n is 3 to 50, X₁ is any amino acid or derivative thereof and is attached to one of the dye or the linker, X₂ is any amino acid, and X3 is lysine or a cysteine derivative attached to the other of the dye or the linker. In some embodiments, X1 is a lysine and comprises an N-terminally blocked amino group.

In some, the peptide comprises a sulfur covalently attached to the dye, and comprises an amine attached to the nucleotide molecule (via a linker moiety). In some embodiments, the peptide has the structure X1-(X2)ₙ-X3, wherein:
n is 3 to 50,
X1 or X3 is a cysteine derivative including a sulfur attached to the nucleotide molecule (via a linker), and
wherein:
   (a) when X1 is the cysteine derivative, X3 or an instance of X2 is a lysine having an amino side chain attached to the dye moiety, or
   (b) when X3 is the cysteine derivative, either: (i) X1 is any amino acid having an N-terminus attached to the dye moiety, or (ii) X1 or an instance of X2 is a lysine having an amino acid chain attached to the dye moiety. In some embodiments, when the X2 is the lysine, X1 comprises a blocked amino group (e.g., acetylated).

In some embodiments, the peptide comprises the sequence K(D/E)nC, wherein n is 4 to 20. In some embodiments, n is 6. In some embodiments, the peptide comprises the sequence KDDDDDDC (SEQ ID NO: 1).

In some embodiments, the peptide comprises an enzymatically cleavable sequence. An enzymatically cleavable sequence may be useful, for example, for cleaving the dye moiety from the nucleotide molecule during a sequencing by synthesis reaction. In some embodiments, the enzymatically cleavable sequence is a protease target amino acid sequence. In some embodiments, the enzymatically cleavable sequence comprises a LVPRGS (SEQ ID NO: 2), which is a thrombin cleavage site. In some embodiments, the peptide comprises ENLYFQG (SEQ ID NO: 3), which is a TEV protease cleavage site. In some embodiments, the peptide comprises DDDDKX (SEQ ID NO: 4), which is an enterokinase cleavage site (cleaving after the lysine).

### Nucleotide of the P-Modified Nucleotide

As previously described, provided herein are modified nucleotide molecules. The nucleotide molecule includes a sugar and a nucleobase.

In some embodiments, the sugar of the nucleotide comprises a ribose. In some embodiments, the sugar comprises a 2′-deoxyribose. In some embodiments, a 5′ phosphoryl group of the sugar comprises a mono-, di-, or tri-phosphate.

In some embodiments, the sugar of the nucleotide comprises a 3′ blocking group. In some embodiments, the P-modified nucleotide molecules are blocked from extension from the 3′ sugar position. In some embodiments, the P-modified nucleotide molecules are reversibly blocked from extension from the 3′ sugar position. In particular embodiments, the 3′ blocking group comprises O-azidomethyl. In some embodiments, sugar of the nucleotide includes a 3′ protecting group. Protecting groups can be used to temporarily block a reactive group. Example protecting groups include: N(6)-benzoyl A, N(4)-benzoyl C, and N(2)-isobutyryl G.

In some embodiments, the base is selected from the group consisting of: an adenine, an analogue of adenine, a cytosine, an analogue of cytosine, a guanine, an analogue of guanine, a thymine, an analogue of thymine, a uracil, and an analogue of uracil. In some embodiments, the base is selected from the group consisting of: an adenine, an analogue of adenine (e.g., 2-aminopurine, which can base pair with T or C), a cytosine, an analogue of cytosine (e.g., G-clamp, which base pairs with guanine), a guanine, an analogue of guanine (e.g., acyclovir), a thymine, and an analogue of thymine (e.g., 5-bromodeoxyuridine).

In some embodiments, the nucleotide molecule includes a 5′ phosphoryl group. In some embodiments, the 5′ phosphoryl group is selected from: a hexaphosphate, a pentaphosphate, a tetraphosphate, a triphosphate, a diphosphate, and a monophosphate. In some embodiments, a 5′ phosphoryl group of the sugar comprises a mono-, di-, or tri-phosphate. In particular embodiments, the 5′ phosphoryl group is a triphosphate. In particular embodiments, the phosphoryl group is a mono-, di-, or tri-phosphate. In particular embodiments, the phosphoryl group is a hexaphosphate.

In some aspects, the nucleotide of the P-modified nucleotide has the structure:

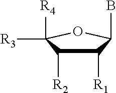

wherein B is a nucleobase, R₁ is H or hydroxy, R₂ is —OR5 or a blocking group, R₃ is a phosphoryl group (e.g., a triphosphate), and R₄ is H.

In particular embodiments, R₂ is —OR₅. In some embodiments, R₅ is selected from the group consisting of: azidomethyl, allyl, methyl, methyl carbamate, hydroxymethyl, amine, ester, disulfide, sulfate, and phosphate. In particular embodiments, R₂ is hydroxy. In particular embodiments, R₅ is a protecting group, such as acetoxime, which serves to protect a 3′-ONH₂ blocking group.

### Linkers

A nucleotide molecule of an P-modified nucleotide molecule as described herein may be directly or indirectly linked to a peptide spacer. In some embodiments, the nucleotide molecule is indirectly attached to the peptide spacer via a linker covalently attaching the peptide to the nucleotide molecule. In some embodiments, a linker includes a moiety generated by conjugating a nucleotide molecule to the peptide spacer.

In some embodiments, the linker is polar and/or charged. In some embodiments, the linker is not non-polar. In some embodiments, the linker is polar. Examples of polar moieties that may be included in the linker include poly(ethylene oxide), poly(propylene oxide), carbamate, ester aldehydes, ketones, and succinimide groups such as thiosuccinimide. In some embodiments, the linker includes one or more poly(ethylene oxide) moieties. In some embodiments, the linker includes one to five poly(ethylene oxide) moieties.

In some embodiments, a linker includes a moiety generated by click chemistry conjugation of a first click chemistry reacting group attached to a nucleotide molecule with a second click chemistry group attached to the peptide spacer. In some instances, the nucleotide molecule and the linker are conjugated via first and second click reactive functional groups using a click reaction. In some embodiments, the first click reactive functional group and second click reactive functional group are selected from: azido/alkynyl groups; alkynyl/azido groups; azido/dibenzocyclooctynyl (DBCO) groups; dibenzocyclooctynyl (DBCO)/azido groups; azido/cyclooctynyl groups; cyclooctynyl/azido groups; tetrazine/dienophile groups; dienophile/tetrazine groups; thiol/alkynyl groups; alkynyl/thiol groups; cyano/1,2-amino thiol groups; 1,2-amino thiol/cyano groups; nitrone/cyclooctynyl groups; cyclooctynyl/nitrone groups; or any combination thereof.

In some instances, the linker includes a cleavable moiety (also referred to as a cleavable linker). In some instances, the cleavable moiety comprises a photocleavable linker, a Pd-cleavable linker, or a reducing agent-cleavable linker such as a phosphine-cleavable linker or a disulfide linker.

In some instances, the cleavable linker includes a photocleavable linker. Any suitable photocleavable linker can be used (see, e.g., Seo et al. (2005), PNAS 102 (17): 5926-5931, which is incorporated by reference herein in its entirety). In some instances, the photocleavable linker comprises a nitrobenzyl group. For instance, a photocleavable nitrobenzyl linker can be cleaved using laser irradiation (e.g., 355 nm, 10 seconds, 1.5 Wcm⁻²).

In some instances, the cleavable linker includes a Pd-cleavable linker. Any suitable Pd-cleavable linker can be used (see, e.g., Ju et al. (2006), PNAS 103 (52): 19635-19640, which is incorporated by reference herein in its entirety). In some instances, the Pd-cleavable linker comprises an allyl group. For instance, a Pd-cleavable allyl linker can be cleaved using incubation with a Na₂PdCl₄/P(PhSO₃Na)₃ mixture (e.g., 30 seconds at 70° C.).

In some instances, the cleavable linker is a reducing agent-cleavable linker. Examples of reducing agents that may be used to cleave a reducible linker include Tris(2-carboxyethyl) phosphine and dithiothreitol (DTT). Examples of reducible moieties that may be included in a linker include disulfide and azidomethyl. In some instances, the cleavable linker includes a phosphine-cleavable linker. Any suitable phosphine-cleavable linker can be used (see, e.g., Guo et al. (2008), PNAS 105 (27): 9145-9150, which is incorporated by reference herein in its entirety). In some instances, the phosphine-cleavable linker comprises an azido group. For instance, a phosphine-cleavable azido linker can be cleaved using incubation with a Tris(2-carboxyethyl) phosphine (TCEP) mixture (e.g., 15 minutes at 65° C.).

In some instances, the cleavable linker includes a disulfide bond. For instance, the disulfide bond can be cleaved using incubation with a reducing agent, such as beta-mercaptoethanol, tris(2-carboxyethyl) phosphine (TCEP), or dithiothreitol (DTT).

### Detectable Labels

Peptide-modified nucleotide molecules provided herein comprise one or more detectable labels. The detectable labels may be distinguishable by means of their differences in fluorescence, Raman spectrum, charge, mass, refractive index, luminescence, length, or any other measurable property.

Detectable labels can be suitable for small scale detection and/or suitable for high-throughput screening. As such, suitable detectable labels include, but are not limited to, radioisotopes, fluorophores, chemiluminescent compounds, bioluminescent compounds, quantum dots, and dyes. Dyes moieties include moieties that emit light in an expected emission pattern. The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some instances, the detectable label is bound to another moiety, for example, a nucleotide or nucleotide analog, and can include a fluorescent, a colorimetric, or a chemiluminescent label.

The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. The label can emit a signal or alter a signal delivered to the label so that the presence or absence of the label can be detected.

In some instances, the detectable label is a dye. Dyes used as detectable labels are capable of absorbing and/or emitting light at specific desired wavelengths. In some instances, the dye is a fluorescent dye. Fluorescent dyes are capable of absorbing and emitting light at specific wavelengths. Examples of molecules that can act as fluorescent dyes include coumarin or coumarin-derivative dye, cyanine or a cyanine-derivative dye, fluorescein or a fluorescein-derivative dye, rhodamine or a rhodamine-derivative dye, or phenoxazine or a phenoxazine-derivative dye. Examples of cyanine or cyanine-derivative dyes include Cyanine 2 (Cy2), Cyanine 5 (Cy5), and Cyanine 3 (Cy3). Examples of rhodamine-derivative dyes include Rhod-2, Rhodamine B, Rhodamine Green™, Rhodamine Red™, Rhodamine Phalloidin, Rhodamine 110, Rhodamine 123, and 5-ROX (carboxy-X-rhodamine). Further examples of fluorescent dyes include 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, CI-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DIA (4-Di-16-ASP), DiD (DilC18 (5)), DIDS, Dil (DilC18 (3)), DiO (DiOC18 (3)), DiR (DilC18 (7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycocrythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycocrythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTOR 11, SYTOR 13, SYTOR 17, SYTOR 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 542, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

In some instances, the dye is a fluorescent dye for example as described, for example, in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). In some instances, a fluorescent label comprises a signaling moiety that conveys information through the fluorescence absorption and/or emission properties of one or more molecules. Non-limiting examples of fluorescence properties comprise fluorescence intensity, fluorescence lifetime, emission spectrum characteristics and energy transfer.

### II. Use of Peptide-Modified Nucleotide Molecules

The disclosed peptide-modified nucleotide molecules may be used in any nucleic acid-based assays performed in cell or tissue samples, such as in situ sequencing of a cell or tissue sample attached to a solid support.

In some embodiments, the sequencing method includes sequencing a template nucleic acid molecule, the sequencing comprising:
(a) contacting a priming strand bound to a template nucleic acid molecule with a polymerase and a first plurality of nucleotides comprising a first peptide-modified nucleotide having the structure N-L-P-D, wherein: N is a nucleotide molecule comprising a 3′ blocked sugar and a base, L is a linker, P is a peptide, and D is a dye, thereby incorporating the peptide-modified nucleotide into a 3′ terminus of the priming strand; and
(b) detecting a presence of the first peptide-modified nucleotide in the priming strand to identify a complementary nucleotide in the template nucleic acid molecule.

In some embodiments, the sequencing method includes sequencing a template nucleic acid molecule by:
a) contacting a priming strand bound to a template nucleic acid molecule with (i) a polymerase and (ii) a first plurality of nucleotide molecules including a peptide-modified (P-modified) nucleotide molecule having the structure N-L-P-D, wherein N is a nucleotide, L is a linker linked to the base of the nucleotide, P is a peptide, and D is a dye, to form a transient complex comprising a 3′ terminus of the priming strand, the template nucleic acid molecule, the polymerase, and the P-modified nucleotide molecule; and
b) detecting a presence of the P-modified nucleotide in the complex to identify a complementary nucleotide in the template nucleic acid molecule.

In some embodiments of the sequencing methods described herein, the 3′ terminal nucleotide of the priming strand is reversibly blocked. In some embodiments, the sequencing method includes disrupting the complex, unblocking the reversibly blocked 3′ terminal nucleotide molecule of the priming strand, and contacting the priming strand bound to the template nucleic acid molecule with a polymerase and a second plurality of peptide-modified nucleotide molecules, thereby incorporating a nucleotide molecule of the second plurality of nucleotide molecules into the priming strand.

In some embodiments, the sequencing method includes exposing the 3′ blocked nucleotide to a deblocking agent. Examples of deblocking agents include UV light exposure, palladium, and reducing agents such as DTT and TCEP. In some embodiments, the blocking group is an O-azido blocking group and the deblocking agent is a reducing agent (e.g., DTT or TCEP).

In some embodiments, the peptide-modified nucleotide includes a cleavage moiety between the nucleotide molecule and the dye, and the method further comprises cleaving the cleavage moiety to release the dye, thereby releasing the dye from the nucleotide molecule.

In some embodiments, the linker comprises a cleavable moiety, and the cleavable moiety is cleavable by exposure to a reducing agent and the method further comprises, after the detecting, contacting the P-modified nucleotide molecule with the reducing agent. Examples of reducing agents include dithiothreitol (DTT), sodium borohydride, hydrogen peroxide, formic acid, and tris-2-carboxyethylphosphine hydrochloride (TCEP). In some embodiments, the reducing agent is DTT. In some embodiments, the reducing agent is TCEP. Examples of cleavable moieties that are cleavable by exposure to a reducing agent includes an o-azido moiety and a disulfide moiety.

In some embodiments, the peptide is cleavable and the method includes, after the detecting, contacting the peptide-modified nucleotide molecule with a protease. Examples of proteases include TEV, thrombin, Enterokinase, and Xa protease. In some embodiments, the peptide is cleavable by a carboxypeptidase (which cleaves the peptide at the C-terminus).

In some embodiments, the method further includes de-blocking the 3′ blocking group and cleaving the peptide or linker of the peptide-modified nucleotide to release the dye. In some embodiments, a cleavable moiety of the linker and the 3′ reversible blocking group are both reducing agent-reactive groups, and the cleaving and/or deblocking includes exposing the P-modified nucleotide molecule to the reducing reagent (e.g., DTT or TCEP). In some embodiments, the cleavable moiety and the 3′ reversible blocking group are both UV-reactive groups, and the cleaving and/or deblocking includes expositing the P-modified nucleotide molecule to UV light.

In some embodiments of in situ sequencing methods disclosed herein, the method includes performing all or a subset of the following steps (in addition to a cycle of sequencing a base using an P-modified nucleotide molecule as described herein): preparing a biological sample (e.g., by fixing, sectioning, embedding, and/or clearing a cell or tissue sample, as described elsewhere herein); and contacting target analytes (e.g., target nucleic acid analytes and/or protein analytes) within the prepared biological sample with target-specific probes, as described elsewhere herein. In some instances, the target-specific probes may comprise, e.g., target-specific linear and/or circularizable nucleic acid probes (e.g., padlock probes) designed to hybridize directly or indirectly to specific target nucleic acid analytes. In some instances, the target-specific linear and/or circularizable nucleic acid probes may optionally comprise primer binding sites and/or target-specific barcode (or identifier) sequences. In some instances, the target-specific probes may comprise, e.g., target-specific antibodies designed to bind to specific target protein analytes, where the antibodies are conjugated to nucleic acid sequences. In some instances, the conjugated nucleic acid sequences may optionally comprise primer binding sites and/or target-specific barcode (or identifier) sequences. In some embodiments, the sequencing method includes performing a reverse transcription reaction (e.g., if the probed target nucleic acid analytes comprise RNA molecules) to create cDNA copies of RNA target molecules.

In some embodiments, the sequencing method includes amplifying the probed target analyte molecules and/or their associated target-specific barcode sequences (e.g., using rolling circle amplification (RCA) in the case that target-specific circularizable probes were used to probe target analyte molecules and/or associated barcode sequences). In some embodiments, the method includes contacting the amplified target nucleic acid analytes and/or associated target-specific barcode sequences with sequencing primers designed to hybridize directly or indirectly to the target nucleic acid analytes and/or their associated target-specific barcode sequences.

In some embodiments, the disclosed sequencing methods (e.g., in situ sequencing methods) include: performing a cyclic series of base-by-base sequencing reactions, where each sequencing cycle comprises:
a) contacting each priming strand bound to a template nucleic acid molecule with a polymerase and an P-modified nucleotide (e.g., at least one P-modified nucleotide or a plurality of different P-modified nucleotides); and
b) detecting a detectable label (e.g., dye) of the P-modified nucleotide molecule to identify a complementary nucleotide in the template nucleic acid molecule.

A primer is generally a single-stranded nucleic acid sequence having a 3′ end that can be used as a substrate for a nucleic acid polymerase in a nucleic acid extension reaction. The sequence of nucleotides added during the extension process is determined by the sequence of the template polynucleotide. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer may in some cases refer to a primer binding sequence.

In some instances, the disclosed methods may further comprise processing optical signals (e.g., fluorescence signals) detected in images (e.g., fluorescence images) acquired during the cyclic series of base-by-base sequencing reactions to detect the presence or absence of complementary detectably labeled P-modified nucleotides in each sequencing cycle at the locations of each of a plurality of template nucleic acid molecules (e.g., the locations corresponding to each of a plurality of target analyte molecules and/or their associated target-specific barcode sequences), thereby enabling inference of the nucleotide sequence of the plurality of template nucleic acid molecules (e.g., the plurality of target analyte molecules and/or associated target-specific barcode sequences). In some instances the detection step may comprise the use of an optical imaging technique (e.g., a fluorescence imaging technique) and real time or post-processing measurement of optical signals (e.g., fluorescence signals or the absence thereof) associated with the presence of a specific P-modified nucleotide molecule covalently coupled to the modified 3′ reversibly terminated nucleotide of the priming strand at a plurality of locations corresponding to a plurality of target analytes distributed throughout the biological sample or tethered to specific locations on a substrate surface (e.g., a flow cell surface).

In some instances, the cyclic series of base-by-base sequencing reactions includes at least 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, or more than 50 cycles of the base-by-base sequencing reaction.

Sequencing reactions using the P-modified nucleotide molecules as described herein may include sequencing-by-synthesis, whereby the P-modified nucleotide is incorporated into a priming strand, or sequencing-by-binding, wherein the P-modified nucleotide is transiently bound to a polymerase and priming strand during detection. In some embodiments, the P-modified nucleotide molecule includes a cleavable moiety in a linker or in the peptide, and the cleavable moiety is cleaved following incorporation and detection of the detectable label. In some embodiments, the P-modified nucleotide molecule includes a cleavable linker between the nucleotide molecule and the peptide spacer, and the linker is cleaved following incorporation and detection of the detectable label. In some embodiments, the P-modified nucleotide molecule includes an enzymatically cleavable sequence in the peptide, and the sequence is enzymatically cleaved following incorporation and detection of the detectable label.

In some embodiments, the sequencing reaction is a sequencing-by-synthesis reaction. In some embodiments, upon contacting each priming strand bound to a template nucleic acid molecule with a polymerase and an P-modified nucleotide, the P-modified nucleotide becomes incorporated into an extended priming strand.

In some instances, the sequencing reaction is a sequencing-by-binding reaction. In some embodiments, the priming strand is blocked from incorporation of the P-modified nucleotide molecule. In some embodiments, the first wash conditions are configured to not disrupt the bound P-modified nucleotide/polymerase/priming strand complex. In some instances, the first wash step may comprise, for example, use of the same buffer used for contacting the primed template nucleic acid with a polymerase and nucleotide molecules (but without the polymerase and nucleotide molecules). In some instances, the first wash buffer may not include KCl and/or may include little to no DMSO. In some instances, the first wash buffer is similar to those used for wash buffers as used in wash steps of a Western blot (e.g., a wash buffer added in a Western blot after binding a primary antibody but washing prior to incubation with a secondary antibody, such as PBST). PBST is a phosphate-buffered saline with a low-concentration of detergent, such as 0.05% to 0.1% Tween.

In some embodiments, each cycle of base-by-base sequencing-by-binding using the P-modified nucleotide molecules described herein further includes a second wash step following the detection step in order to disrupt the complex. In some instances, the second wash is performed under more stringent conditions than the first wash. For example, the second wash may include a temperature higher than room temperature (e.g., 30-40° C.), a higher salt concentration (e.g., a higher KCl salt concentrations (e.g., at least 50 mM KCl)), a solvent miscible in the wash buffer solution (e.g., dimethyl sulfoxide (DMSO)), a detergent (e.g., sodium dodecyl sulfate (SDS)), or a combination thereof.

In some instances, the (non-covalently bound) complex consisting of the 3′ terminus of the priming strand, the template nucleic acid molecule, a polymerase, and an P-modified nucleotide molecule may comprise a transient complex. In some instances, the transient complex may persist for at least 5 sec, 10 sec, 20 sec, 30 sec, 40 sec, 50 sec, 1 min, 2 min, 3 min, 4 min, 5 min, or 10 min after removal of polymerase and P-modified nucleotides used to contact the primed template nucleic acid molecule and form the complex. The “persistence time” of the complex, as used herein, refers to the average length of time that the complex remains stable without significant dissociation of any of the components of the bound complex.

In some instances, the sequencing reaction comprises repeating one or more sequencing cycles. In some instances, the sequencing reaction comprises repeating the same sequencing cycle.

In some instances, a sequencing cycle of the sequencing includes contacting the template with a plurality of P-modified nucleotide molecules including first P-modified nucleotide molecules having a first base type and a first dye, second P-modified nucleotide molecules having a second base type and a second dye, third P-modified nucleotide molecules having a third base type and a third dye, and fourth nucleotide molecules having a fourth base type, wherein the first, second, and third dyes are each different. In some instances, the fourth nucleotide molecules are fourth P-modified nucleotide molecules. In some instances, the fourth nucleotide molecules are not linked to a dye. In some embodiments, the fourth nucleotide molecules are linked to a fourth dye that is different from the first, second, and third dyes.

In some embodiments, a sequencing cycle comprises contacting one or more templates with a mixture of nucleotide molecules comprising: (i) first P-modified nucleotide molecules having a first base type and a first dye; (ii) second P-modified nucleotide molecules having a second base type and a second dye; (iii) third P-modified nucleotide molecules having a third base type and a third dye; and (iv) fourth P-modified nucleotide molecules having a fourth base type and a fourth dye, wherein the first, second, third, and fourth bases types are different (e.g., A, T/U, C, and G) and the first, second, third, and fourth dyes are different (e.g., each detectable in a different color channel using fluorescence microscopy).

In some embodiments, a sequencing cycle comprises contacting one or more templates with a mixture of nucleotide molecules comprising: (i) first P-modified nucleotide molecules having a first base type and a first dye; (ii) second P-modified nucleotide molecules having a second base type and a second dye; (iii) third P-modified nucleotide molecules having a third base type and a third dye; and (iv) fourth P-modified nucleotide molecules having a fourth base type, wherein the first, second, third, and fourth bases types are different (e.g., A, T/U, C, and G) and the first, second, and third dyes are different (e.g., each detectable in a different color channel using fluorescence microscopy). In some embodiments, the fourth P-modified nucleotide molecules are not detectably labeled or are detectably labeled but are not detected.

In some embodiments, a sequencing cycle comprises contacting one or more templates with a mixture of nucleotide molecules comprising: (i) first P-modified nucleotide molecules each having the same first base type and being dual labeled or configured to be dual labeled with a first dye and a second dye; (ii) second P-modified nucleotide molecules each having the same second base type and the first dye (but no second dye); (iii) third P-modified nucleotide molecules each having the same third base type and the second dye (but no first dye); and (iv) fourth P-modified nucleotide molecules each having the same fourth base type and no first dye or second dye, wherein the first, second, third, and fourth bases types are different (e.g., A, T/U, C, and G) and the first and second dyes are different (e.g., each detectable in a different color channel using fluorescence microscopy).

In some embodiments, the sequencing reaction involves a repeating pattern of a sequencing cycle comprising two separate steps: a first step and a second step. In some embodiments, the first step comprises contacting one or more templates with a mixture of nucleotide molecules comprising: (i) first P-modified nucleotide molecules each having the same first base type and a dye linked to the first P-modified nucleotide molecule, where the linkage is cleavable; (ii) second P-modified nucleotide molecules each having the same second base type and the dye linked to the second P-modified nucleotide molecule, where the linkage is not cleavable or cleavable but not cleaved in the second step; (iii) third P-modified nucleotide molecules each having the same third base type and a binding moiety configured to bind to the dye but is not bound to the dye in the first step; and (iv) fourth P-modified nucleotide molecules each having the same fourth base type and no dye linked thereto and no binding moiety configured to bind to the dye, wherein the first, second, third, and fourth bases types are different (e.g., A, T/U, C, and G). In some embodiments, the second step comprises: cleaving the dyes linked to the first P-modified nucleotide molecules, wherein the linkage between the dyes and the second P-modified nucleotide molecules is not cleavable or cleavable but not cleaved; contacting the third P-modified nucleotide molecules with the dye to allow it to bind to the binding moiety, thereby labeling the third P-modified nucleotide molecules.

In some instances, the sequencing reaction involves a repeating pattern of two different sequencing cycles. In some embodiments, a first of the two repeating cycles includes contacting the template with a first plurality of P-modified nucleotide molecules including first P-modified nucleotide molecules having a first base type and a first dye and a second P-modified nucleotide molecules having a second base type and a second dye that is different from the first dye. In some embodiments, a second of the two repeating cycles includes contacting the template with a second plurality of P-modified nucleotide molecules including a third P-modified nucleotide molecules having a third base type and a third dye, and fourth P-modified nucleotide molecules having a fourth base type and a fourth dye that is different from the third dye.

In some instances, the sequencing reaction involves a repeating pattern of four different sequencing cycles. In some embodiments, a first of the four repeating cycles includes contacting the template with first P-modified nucleotide molecules having a first base type and a dye, a second of the four repeating cycles includes contacting the template with second P-modified nucleotide molecules having a second base type and a dye, a third of the four repeating cycles includes contacting the template with third P-modified nucleotide molecules having a third base type and a dye, and fourth of the four repeating cycles includes contacting the template with fourth P-modified nucleotide molecules having a fourth base type and a dye.

In some instances, the first plurality of P-modified nucleotide molecules (or one or more additional pluralities of P-modified nucleotide molecules) comprises a set of four different P-modified nucleotide molecules, where each different P-modified nucleotide molecule (e.g., each P-modified nucleotide molecule comprising a different nucleobase) is coupled to a different fluorophore.

In some instances, the first plurality of P-modified nucleotide molecules (or one or more additional pluralities of P-modified nucleotide molecules) comprises a set of four different P-modified nucleotide molecules, where three of the four different P-modified nucleotide molecules are coupled to different fluorophores and one of the four different P-modified nucleotide molecules is not conjugated to a fluorophore.

In some instances, the first plurality of P-modified nucleotide molecules (or one or more additional pluralities of P-modified nucleotide molecules) comprises a set of four different P-modified nucleotide molecules, where two of the four different P-modified nucleotides are coupled to different fluorophores, one of the four different P-modified nucleotide molecules is coupled to the two different fluorophores, and one of the four different P-modified nucleotide molecules is not conjugated to a fluorophore.

In some instances, the first plurality of P-modified nucleotides (or one or more additional pluralities of P-modified nucleotide molecules) are selected from modified A, T, U, C, and G. In some instances, the first plurality of P-modified nucleotides (or one or more additional pluralities of P-modified nucleotide molecules) are selected from modified A, T, C, and G.

In some instances, the first plurality of P-modified nucleotide molecules and at least one additional plurality of P-modified nucleotide molecules may comprise a same set of P-modified nucleotide molecules (e.g., P-modified nucleotides comprising the same set of nucleobases). In some instances, the first plurality of P-modified nucleotide molecules and at least one additional plurality of P-modified nucleotide molecules may comprise different sets of P-modified nucleotide molecules (e.g., P-modified nucleotides comprising different sets of nucleobases).

In some instances, the first plurality of P-modified nucleotide molecules and at least one additional plurality of P-modified nucleotide molecules may each comprise P-modified nucleotide molecules that do not include a 3′ reversible terminator moiety.

In some instances, the first plurality of P-modified nucleotide molecules and at least one additional plurality of P-modified nucleotide molecules may each comprise at least one P-modified nucleotide molecule that is not labeled with a detectable label.

Methods for processing the series of optical signals detected over the course of performing a cyclic series of base-by-base sequencing reactions to identify a nucleotide sequence are described elsewhere herein.

Examples of polymerases that may be used for performing the disclosed methods include, but are not limited to, DNA polymerases (e.g., Taq DNA polymerase), RNA polymerases, and/or reverse transcriptases.

In some aspects, the polymerase is a DNA polymerase. In some embodiments, the polymerase is a DNA polymerase, the template nucleic acid comprises DNA, and the peptide-modified nucleotide molecules are DNA molecules. Examples of DNA polymerases include Taq polymerase, 9°N-7 DNA polymerase (or variants thereof, for example, D141A/E143A/A485L), phi29 (φ29) polymerase, Klenow fragment, Bacillus stearothermophilus DNA polymerase (BST), T4 DNA polymerase, T7 DNA polymerase, and DNA polymerase I. In some aspects, the polymerase is phi29 DNA polymerase. In some aspects, the polymerase of the polymerase conjugate is a DNA polymerase and the template nucleic acid molecule includes DNA. In some aspects, the polymerase is a DNA polymerase, the P-modified nucleotide molecules include P-modified nucleotide molecules, and the template comprises DNA. In some aspects, the template comprises cDNA.

In some instances, the polymerase is selected from Taq polymerase, a family B polymerase such as 9°N-7 DNA polymerase or a functional variant thereof (e.g., D141A/E143A/A485L), and a Klenow fragment of DNA polymerase I. In some aspects, the DNA polymerase is Taq polymerase or a functional variant thereof. Taq polymerase is a heat-stable polymerase from Thermus aquaticus. In some aspects, the DNA polymerase is phi29 DNA polymerase or a functional variant thereof. The DNA polymerase of phi29 (a phage of Bacillus subtilis) has high processivity and fidelity. In some aspects, the DNA polymerase is a 9°N-7 DNA polymerase or a functional variant thereof (e.g., D141A/E143A/A485L In some aspects, the DNA polymerase is DNA polymerase I or a functional fragment thereof (e.g., a Klenow fragment). Klenow fragment is an exonuclease deficient fragment of DNA polymerase I.

In some aspects, the polymerase is a reverse transcriptase. Reverse transcriptases can have RNA-dependent DNA polymerase activity and DNA-dependent DNA polymerase activity. In some embodiments, the polymerase is a reverse transcriptase, the template nucleic acid molecule comprises RNA, and the peptide-modified nucleotide molecules are RNA molecules. Examples of reverse transcriptases include Moloney murine leukemia virus (MMLV) reverse transcriptase, HIV-1 reverse transcriptase, and avian myeloblastosis virus (AMV) reverse transcriptase. In some aspects, the reverse transcriptase lacks (e.g., is mutated to lack) ribonuclease activity. In some instances, ribonuclease activity may degrade the template particularly during longer incubation times such as when reverse transcribing longer cDNAs.

In some embodiments, the priming strand comprises a 3′ terminal nucleotide that is reversibly blocked. Thus, the P-modified nucleotide does not become incorporated into the priming strand during formation of the complex. In some embodiments, the method further comprises the additional steps of: disrupting the complex, unblocking the reversibly blocked 3′ terminal nucleotide molecule of the priming strand, and contacting the priming strand bound to the template nucleic acid molecule with a polymerase and a second plurality of nucleotide molecules, thereby incorporating a nucleotide molecule of the second plurality of nucleotide molecules into the priming strand. In some embodiments, the method further includes repeating a cycle of steps (a) and (b) and the additional steps of disrupting the complex for at least one additional cycle, thereby identifying an additional complementary nucleotide in the template nucleic acid molecule. In some embodiments, the method includes repeating the cycle for at least 2, 5, 10, 20, or 30 additional cycles.

In some embodiments, the priming strand comprises a 3′ terminal nucleotide that is unblocked, and wherein step (a) further comprises incorporating the P-modified nucleotide into the priming strand. In some embodiments, the P-modified nucleotide comprises a reversibly blocked 3′ position, and the method further comprises, after the incorporating, unblocking the reversibly blocked 3′ position. In some embodiments, the linker comprises a cleavable moiety, and the method further comprises: (c) cleaving the cleavable moiety to release the dye and the peptide from the peptide-modified nucleotide. In some embodiments, the cleavable moiety is photocleavable, and the cleaving comprises exposing the P-modified nucleotide to UV light, or the cleavable moiety is a disulfide or an O-azido moiety, and the cleaving comprises contacting the P-modified nucleotide with a reducing agent. In some embodiments, the method further includes repeating a cycle steps (a)-(c), thereby incorporating an additional P-modified nucleotide into the priming strand and identifying an additional complementary nucleotide in the template nucleic acid molecule. In some embodiments, the repeating is for at least 2, at least 5, at least 10, at least 20, or at least 30 additional cycles.

In some embodiments of any of the sequencing methods provided herein, the template nucleic acid molecule comprises a DNA molecule. In some embodiments, the template nucleic acid molecule comprises an RNA molecule, optionally wherein the RNA molecule is an mRNA molecule. In some embodiments, the template nucleic acid molecule comprises a target analyte nucleic acid molecule. In some embodiments, the template nucleic acid molecule comprises a barcode sequence associated with a target analyte.

In some embodiments, the sequencing methods further include hybridizing a circularizable probe or probe set to the target analyte or to a labeling agent bound to the target analyte and ligating the circularizable probe or probe set to form a circularized probe, wherein the method further comprises performing rolling circle amplification of the circularized probe to generate the template nucleic acid molecule. In some embodiments, the circularizable probe or probe set is a padlock probe. In some embodiments, the template nucleic acid molecule to be sequenced is attached to a solid support. In some embodiments, the template nucleic acid molecule is sequenced in situ in a cell sample or tissue sample. In some embodiments, the cell sample comprises a layer of cells deposited on a surface.

In some aspects, provided are sequencing methods using P-modified nucleotide molecules as described herein. The sequencing methods include multi-cycle sequencing approaches where a cyclic series of steps are performed to identify nucleotides base-by-base in a template nucleic acid sequence (e.g., a target analyte sequence and/or an associated target-specific barcode sequence).

In some instances, the template nucleic acid molecule includes a target analyte nucleic acid molecule (e.g., a DNA molecule, an RNA molecule, or an mRNA molecule). In some instances, the template nucleic acid includes a reporter oligonucleotide, such as a barcode.

In some instances, the template nucleic acid molecule comprises DNA. Examples of DNA template nucleic acid molecules include DNA molecules such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA molecules can be copies from another nucleic acid molecule (e.g., DNA or RNA such as mRNA).

In some instances, the methods includes contacting a template nucleic acid molecule with a sequencing primer designed to hybridize to a portion of the template nucleic acid molecule, where the sequencing primer includes a reversibly terminated nucleotide at its 3′ end. In such embodiments, the modified, 3′ reversibly terminated nucleotide blocks incorporation of P-modified nucleotide molecules into the sugar-phosphate. Such sequencing primers may be used in a sequencing-by-binding approach using the P-modified nucleotide molecules described herein.

In some instances, the methods include contacting a template nucleic acid molecule with a sequencing primer designed to hybridize to a portion of the template nucleic acid molecule, where the sequencing primer does not include a reversibly terminated nucleotide at its 3′ end. In such embodiments, an P-modified nucleotide molecule is incorporated into the sugar-phosphate of the priming strand. Such sequencing primers may be used in a sequencing-by-synthesis approach using the P-modified nucleotide molecules described herein.

### Biological Samples

In some aspects, provided herein are methods of sequencing nucleic acids obtained from a biological sample. A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1 μm) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

In some instances, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some instances, the biological sample is prepared using formalin-fixation and paraffin-embedding (FFPE), which are established methods. In some instances, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some instances, the methods provided herein include one or more post-fixing (also referred to as post-fixation) steps. In some instances, one or more post-fixing step is performed after contacting a sample with a polynucleotide disclosed herein, e.g., one or more probes such as a circular or padlock probe. In some instances, one or more post-fixing step is performed after a hybridization complex comprising a probe and a target is formed in a sample. In some instances, one or more post-fixing step is performed prior to a ligation reaction disclosed herein.

In some instances, a method disclosed herein includes de-crosslinking the reversibly cross-linked biological sample. The de-crosslinking does not need to be complete. In some instances, only a portion of crosslinked molecules in the reversibly cross-linked biological sample are de-crosslinked and allowed to migrate.

In some instances, a biological sample can be permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some instances, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some instances, the biological sample is permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some instances, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

In some instances, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some instances, the hydrogel is formed such that the hydrogel is internalized within the biological sample. Biological samples can include analytes (e.g., protein, RNA, and/or DNA) embedded in a 3D matrix. In some instances, amplicons (e.g., rolling circle amplification products) derived from or associated with analytes (e.g., protein, RNA, and/or DNA) can be embedded in a 3D matrix. In some instances, a 3D matrix may comprise a network of natural molecules and/or synthetic molecules that are chemically and/or enzymatically linked, e.g., by crosslinking. In some instances, a 3D matrix may comprise a synthetic polymer. In some instances, a 3D matrix comprises a hydrogel.

In some aspects, a biological sample is embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some instances, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some instances, one or more of the polynucleotide probe(s) and/or amplification product (e.g., amplicon) thereof can be modified to contain functional groups that can be used as an anchoring site to attach the polynucleotide probes and/or amplification product to a polymer matrix. In some instances, a modified probe comprising oligo dT may be used to bind to mRNA molecules of interest, followed by reversible or irreversible crosslinking of the mRNA molecules.

In some instances, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some instances, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly (hydroxyethyl acrylate), and poly (hydroxyethyl methacrylate), collagen, hyaluronic acid, chitosan, dextran, agarose, gelatin, alginate, protein polymers, methylcellulose, and the like, and combinations thereof.

In some instances, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347 (6221): 543-548, 2015, the entire contents of which are incorporated herein by reference.

In some instances, the hydrogel forms the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some instances, hydrogel formation occurs after contacting one or more second materials during formation of the substrate. Hydrogel formation can also occur within a structure (e.g., wells, ridges, projections, and/or markings) located on a substrate.

In some instances, hydrogel formation on a substrate occurs before, contemporaneously with, or after probes are provided to the sample. For example, hydrogel formation can be performed on the substrate already containing the probes.

In some instances, hydrogel formation occurs within a biological sample. In some embodiments, a biological sample (e.g., tissue section) is embedded in a hydrogel. In some instances, hydrogel subunits are infused into the biological sample, and polymerization of the hydrogel is initiated by an external or internal stimulus.

In instances in which a hydrogel is formed within a biological sample, functionalization chemistry can be used. In some instances, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and cPACT. In some instances, hydrogel formation within a biological sample is permanent. For example, biological macromolecules can permanently adhere to the hydrogel allowing multiple rounds of interrogation. In some instances, hydrogel formation within a biological sample is reversible. In some instances, HTC reagents are added to the hydrogel before, contemporaneously with, and/or after polymerization. In some instances, a cell labeling agent is added to the hydrogel before, contemporaneously with, and/or after polymerization. In some instances, a cell-penetrating agent is added to the hydrogel before, contemporaneously with, and/or after polymerization.

In some instances, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some instances, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some instances, a hydrogel-embedded sample is stored before or after clearing of hydrogel, in a medium (e.g., a mounting medium, methylcellulose, or other semi-solid mediums).

In some instances, a biological sample embedded in a matrix (e.g., a hydrogel) is isometrically expanded. Isometric expansion methods that can be used include hydration, a preparative step in expansion microscopy, as described in, e.g., Chen et al., Science 347 (6221): 543-548, 2015 and U.S. Pat. No. 10,059,990, which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some instances, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some instances, the sample is isometrically expanded to at least 2× and less than 20× of its non-expanded size.

To facilitate visualization, biological samples can be stained using a wide variety of stains and staining techniques. In some instances, for example, a sample can be stained using any number of stains and/or immunohistochemical reagents. One or more staining steps may be performed to prepare or process a biological sample for an assay described herein or may be performed during and/or after an assay. In some instances, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, SSDNA), RNA, an organelle or compartment of the cell. The sample may be contacted with one or more labeled antibodies (e.g., a primary antibody specific for the analyte of interest and a labeled secondary antibody specific for the primary antibody). In some instances, cells in the sample can be segmented using one or more images taken of the stained sample.

In some instances, the stain is performed using a lipophilic dye. In some examples, the staining is performed with a lipophilic carbocyanine or aminostyryl dye, or analogs thereof (e.g., Dil, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some instances, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some instances, the sample can be stained using Romanowsky stain, including Wright's stain, Jenner's stain, Can-Grunwald stain, Leishman stain, and Giemsa stain.

In some instances, biological samples can be destained. Any suitable methods of destaining or discoloring a biological sample may be utilized and generally depend on the nature of the stain(s) applied to the sample. For example, in some instances, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65 (8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some aspects, provided herein are sequencing methods for sequencing a template nucleic acid molecule. In some embodiments, the template nucleic acid molecule to be sequenced is attached to a solid support.

In some embodiments, a template nucleic acid molecule is sequenced in situ in a cell sample or tissue sample. In some embodiments, the cell or tissue sample is attached to a solid support. In some embodiments, the cell sample includes a layer of cells deposited on a surface. In some embodiments, the tissue sample is an formalin fixed paraffin embedded tissue sample processed for in situ sequencing. In some embodiments, the tissue sample is fresh frozen tissue sample processed for in situ sequencing.

A sample disclosed herein can be or derived from any biological sample. The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, a cell pellet, a cell block, a needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some instances, the biological sample may be provided on a substrate. In some instances, a substrate herein can be any support that is insoluble in aqueous liquid and which allows for positioning of biological samples, analytes, features, and/or reagents (e.g., probes) on the support. In some instances, a biological sample can be attached to a substrate. Attachment of the biological sample can be irreversible or reversible, depending upon the nature of the sample and subsequent steps in the analytical method. In certain instances, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some instances, the substrate can be coated or functionalized with one or more substances to facilitate attachment of the sample to the substrate.

Suitable substances that can be used to coat or functionalize the substrate include, but are not limited to, lectins, poly-lysine, antibodies, and polysaccharides.

A biological sample may comprise one or a plurality of analytes of interest. Methods for performing multiplexed assays to analyze two or more different analytes in a single biological sample are provided.

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of an RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or P-modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA. In particular embodiments, the analyte includes RNA. In particular embodiments, the analyte includes RNA and/or DNA, and the sequencing method includes determining the presence of a single nucleotide polymorphism.

In some embodiments, an analyte herein is endogenous to a biological sample and can include nucleic acid analytes and non-nucleic acid analytes. Methods and compositions disclosed herein can be used to analyze nucleic acid analytes.

In some instances, provided herein are methods and compositions for analyzing endogenous analytes (e.g., RNA, ssDNA, cell surface or intracellular proteins, and/or metabolites) in a sample using one or more labeling agents. In some instances, an analyte labeling agent may include an agent that interacts with an analyte (e.g., an endogenous analyte in a sample). In some instances, the labeling agents can comprise a reporter oligonucleotide that is indicative of the analyte or portion thereof interacting with the labeling agent. For example, the reporter oligonucleotide may comprise a barcode sequence that permits identification of the labeling agent. In some instances, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes a barcode that is associated with or otherwise identifies the analyte binding moiety. In some instances, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some instances, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some instances, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a DARPin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of non-limiting examples of labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some instances, an analyte binding moiety includes one or more antibodies or epitope-binding fragments thereof. The antibodies or epitope-binding fragments including the analyte binding moiety can specifically bind to a target analyte. In some instances, the analyte is a protein (e.g., a protein on a surface of the biological sample (e.g., a cell) or an intracellular protein). In some instances, a plurality of analyte labeling agents comprising a plurality of analyte binding moieties bind a plurality of analytes present in a biological sample. In some instances, the plurality of analytes includes a single species of analyte (e.g., a single species of polypeptide). In some instances in which the plurality of analytes includes a single species of analyte, the analyte binding moieties of the plurality of analyte labeling agents are the same. In some instances in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some instances, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31 (2): 708-715, which is entirely incorporated herein by reference for all purposes. Likewise, protein and peptide biotinylation techniques have been developed and are readily available. Sec, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some instances, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some instances, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

In some instances, provided herein are methods and compositions for analyzing one or more products of an endogenous analyte and/or a labeling agent in a biological sample. In some instances, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some instances, a labeling agent that directly or indirectly binds to an analyte in the biological sample is analyzed. In some instances, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed.

In some instances, a hybridization product comprising the pairing of substantially complementary or complementary nucleic acid sequences within two different molecules can be analyzed. For example, hybridization of an endogenous analyte or the labeling agent (e.g., reporter oligonucleotide attached thereto) with another endogenous molecule or another labeling agent or a probe can be analyzed. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labeling agent and each probe may comprise one or more barcode sequences. Non-limiting examples of barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

In some aspects, sequencing methods provided herein (e.g., in situ sequencing methods) include a ligation step. Ligations are usually carried out enzymatically to form a phosphodiester linkage between a 5′ terminal nucleotide with a 3′ terminal nucleotide.

In some embodiments, a ligation product of an endogenous analyte and/or a labeling agent is analyzed. In some instances, the ligation product is formed between two or more endogenous analytes. In some instances, the ligation product is formed between two or more labeling agents. In some instances, the ligation product is an intramolecular ligation of an endogenous analyte. In some instances, the ligation product is an intramolecular ligation product or an intermolecular ligation product, for example, the ligation product can be generated by the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some instances, sequencing methods included herein include use of a probe or probe set capable of DNA-templated ligation, such as from a cDNA molecule. Sec, e.g., U.S. Pat. No. 8,551,710, which is hereby incorporated by reference in its entirety. In some instances, sequencing methods included herein include use of a probe or probe set capable of RNA-templated ligation. Sec, e.g., U.S. Pat. Pub. 2020/0224244 which is hereby incorporated by reference in its entirety. In some instances, the probe set is a SNAIL probe set. See, e.g., U.S. Pat. Pub. 20190055594, which is hereby incorporated by reference in its entirety. In some instances, provided herein is a multiplexed proximity ligation assay. Sec, e.g., U.S. Pat. Pub. 20140194311 which is hereby incorporated by reference in its entirety. In some instances, sequencing methods included herein include use of a probe or probe set capable of proximity ligation, for instance a proximity ligation assay for RNA (e.g., PLAYR) probe set. Sec, e.g., U.S. Pat. Pub. 20160108458, which is hereby incorporated by reference in its entirety. In some instances, a circular probe is indirectly hybridized to the target nucleic acid. In some instances, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some instances, the ligation involves chemical ligation (e.g., click chemistry ligation). In some instances, the chemical ligation involves template dependent ligation. In some instances, the chemical ligation involves template independent ligation. In some instances, the click reaction is a template-independent reaction (see, e.g., Xiong and Seela (2011), J. Org. Chem. 76 (14): 5584-5597, incorporated by reference herein in its entirety). In some instances, the click reaction is a template-dependent reaction or template-directed reaction. In some instances, the template-dependent reaction is sensitive to base pair mismatches such that reaction rate is significantly higher for matched versus unmatched templates. In some instances, the click reaction is a nucleophilic addition template-dependent reaction. In some instances, the click reaction is a cyclopropane-tetrazine template-dependent reaction.

In some instances, the ligation involves an enzymatic ligation. In some instances, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some instances, the ligase is a T4 RNA ligase. In some instances, the ligase is a splintR ligase. In some instances, the ligase is a single stranded DNA ligase. In some instances, the ligase is a T4 DNA ligase. In some instances, the ligase is a ligase that has an DNA-splinted DNA ligase activity. In some instances, the ligase is a ligase that has an RNA-splinted DNA ligase activity.

In some instances, the ligation herein is a direct ligation. In some instances, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some instances, said ends are not ligated directly to each other, but instead occurs either via the intermediacy of one or more intervening (so-called “gap” or “gap-filling” (oligo) nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo) nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific instances, the gap may be a gap of about 1, 2, 3, 4, 5, 6, 7, 8, 9 or 10 or more nucleotides, of any integer (or range of integers) of nucleotides in between the indicated values. In some instances, the gap between said terminal regions may be filled by a gap oligonucleotide or by extending the 3′ end of a polynucleotide. In some cases, ligation involves ligating the ends of the probe to at least one gap (oligo) nucleotide, such that the gap (oligo) nucleotide becomes incorporated into the resulting polynucleotide. In some instances, the ligation herein is preceded by gap filling. In other instances, the ligation herein does not require gap filling.

In some instances, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some instances, the ligation herein is a proximity ligation of ligating two (or more) nucleic acid sequences that are in proximity with each other, e.g., through enzymatic means (e.g., a ligase). In some instances, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

In some instances, the sequencing methods provided herein including analyzing a primer extension product of an analyte, a labeling agent, a probe or probe set bound to the analyte (e.g., a circularizable probe bound to genomic DNA, mRNA, or cDNA), or a probe or probe set bound to the labeling agent (e.g., a circularizable probe bound to one or more reporter oligonucleotides from the same or different labeling agents).

A primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some instances, a product of an endogenous analyte and/or a labeling agent is an amplification product of one or more polynucleotides, for instance, a circular probe or circularizable probe or probe set. In some instances, the amplifying is achieved by performing rolling circle amplification (RCA). In other instances, a primer that hybridizes to the circular probe or circularized probe is added and used as such for amplification. In some instances, the RCA comprises a linear RCA, a branched RCA, a dendritic RCA, or any combination thereof.

In some instances, the amplification is performed at a temperature between or between about 20° C. and about 60° C. In some instances, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some instances, upon addition of a DNA polymerase in the presence of appropriate dNTP precursors and other cofactors, a primer is elongated to produce multiple copies of the circular template. This amplification step can utilize isothermal amplification or non-isothermal amplification. In some instances, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49 (11): 2540-2550; Schweitzer et al. Proc. Natl Acad. Sci. USA 97:10113-119, 2000; Faruqi et al, BMC Genomics 2:4, 2000; Nallur et al, Nucl. Acids Res. 29: e118, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801). Non-limiting examples of polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some instances, the polymerase is phi29 DNA polymerase.

In some aspects, during the amplification step, P-modified nucleotides can be added to the reaction to incorporate the P-modified nucleotides in the amplification product (e.g., nanoball). Non-limiting examples of the P-modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprise a P-modified nucleotide, such as an amine-modified nucleotide. In some instances, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N⁶-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some instances, one or more of the polynucleotide probe(s) can be modified to contain functional groups that can be used as an anchoring site to attach the polynucleotide probes and/or amplification product to a polymer matrix. Non-limiting examples of modification and polymer matrix that can be employed in accordance with the provided instances comprise those described in, for example, WO 2014/163886, WO 2017/079406, US 2016/0024555, US 2018/0251833 and US 2017/0219465, which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some instances, the provided methods involve embedding the one or more polynucleotide probe sets and/or the amplification products in the presence of hydrogel subunits to form one or more hydrogel-embedded amplification products. In some instances, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some instances, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some instances, the RCA template may comprise the target analyte, or a part thereof, where the target analyte is a nucleic acid, or it may be provided or generated as a proxy, or a marker, for the analyte. In some instances, different analytes are detected in situ in one or more cells using a RCA-based detection system, e.g., where the signal is provided by generating an RCA product from a circular RCA template which is provided or generated in the assay, and the RCA product is detected to detect the corresponding analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some instances, a product herein includes a molecule or a complex generated in a series of reactions, e.g., hybridization, ligation, extension, replication, transcription/reverse transcription, and/or amplification (e.g., rolling circle amplification), in any suitable combination.

### Fluorescence Detection and Imaging

As previously described, provided herein are P-modified nucleotide molecules that include a dye moiety, such as a fluorophore. Methods of using the P-modified nucleotide molecules include detection of the dye to detect the presence of the P-modified nucleotide molecule, such as for in situ sequencing of a cell or tissue sample. Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some instances, methods disclosed herein provide surprisingly reduced tissue autofluorescence.

Examples of fluorescent labels and nucleotides and/or polynucleotides conjugated to such fluorescent labels comprise those described elsewhere herein and those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some instances, non-limiting examples of techniques and methods applicable to the provided embodiments comprise those described in, for example, U.S. Pat. Nos. 4,757,141, 5,151,507 and 5,091,519.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some instances, fluorescence microscopy is used for detection and imaging of the sample. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be or comprise any microscope that uses fluorescence to generate an image, whether it is a simpler set up like an epifluorescence microscope, or a more complicated design such as a confocal microscope, which uses optical sectioning to achieve better z-axis resolution of the sample to be imaged.

In some instances, confocal microscopy is used for detection and imaging of the sample. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity-so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immune-stained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECS™), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PS™, photon scanning tunneling microscopy (PS™), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXS™), and intact tissue expansion microscopy (exM).

In some instances, a method herein comprises subjecting the sample to expansion microscopy methods and techniques. Expansion allows individual targets (e.g., mRNA or RNA transcripts) which are densely packed within a cell, to be resolved spatially in a high-throughput manner. Expansion microscopy techniques are known in the art and can be performed as described in US 2016/0116384 and Chen et al., Science, 347, 543 (2015), each of which are incorporated herein by reference in their entirety. In some instances, the method does not comprise subjecting the sample to expansion microscopy. In some instances, the method does not comprise dissociating a cell from the sample such as a tissue or the cellular microenvironment. In some instances, the method does not comprise lysing the sample or cells therein. In some instances, the method does not comprise embedding the sample or molecules from the sample in an exogenous matrix.

In some cases, analysis is performed on one or more images captured, and may comprise processing the image(s) and/or quantifying signals observed. In some instances, images of signals from different fluorescent channels and/or nucleotide incorporation cycles can be compared and analyzed. In some instances, images of signals (or absence thereof) at a particular location in a sample from different fluorescent channels and/or sequential incorporation cycles can be aligned to analyze an analyte at the location. For instance, a particular location in a sample can be tracked and signal spots from sequential incorporation cycles can be analyzed to detect a target polynucleotide sequence (e.g., a barcode sequence or subsequence thereof) in an analyte at the location. The analysis may comprise processing information of one or more cell types, one or more types of analytes, a number or level of analyte, and/or a number or level of cells detected in a particular region of the sample. In some instances, the analysis comprises detecting a sequence e.g., a barcode sequence present in an amplification product at a location in the sample. In some instances, the number of signals detected in a unit area in the biological sample is quantified. In some instances, the signals detected at a corresponding position in the biological sample in a plurality of images taken at different z positions (e.g., in the depth direction) are quantified and analyzed.

In some instances, sequencing methods disclosed herein (e.g., in situ sequencing methods) include sequencing a barcode sequence. Analytes described herein can be associated with one or more barcode(s), e.g., at least two, three, four, five, six, seven, eight, nine, ten, or more barcodes. Barcodes can be used to spatially-resolve molecular components found in biological samples, for example, within a cell or a tissue sample. A barcode can be attached to an analyte or to another moiety or structure (e.g., a target-specific antibody) in a reversible or irreversible manner. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some instances, a barcode includes two or more sub-barcodes (or barcode segments) that together function as a single barcode. For example, a polynucleotide barcode can include two or more polynucleotide sequences (e.g., sub-barcodes) that are contiguous or that are separated by one or more non-barcode sequences. In some instances, a barcode may comprise about 1, 2, 3, 4, 5, 6, 7, 8, 9, 10, or more than 10 sub-barcodes (or barcode segments). In some instances, each sub-barcode (or barcode segment) may comprise about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides. In some instances, each non-barcode sequence may comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some instances, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide. In any of the preceding instances, the methods provided herein can include analyzing the barcodes performing in situ sequencing using the P-modified nucleotide molecules as disclosed herein.

In some instances, e.g., in a barcode sequencing method, barcode sequences are detected for identification of other molecules including nucleic acid molecules (DNA or RNA) that are longer than the barcode sequences themselves, as opposed to direct sequencing of the longer nucleic acid molecules. In some instances, an N-mer barcode sequence can comprise up to 4^{N} unique sequences given a sequencing read of N bases, and a much shorter sequencing read may be required for molecular identification compared to non-barcoded sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some instances, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub 20210164039, which are hereby incorporated by reference in their entirety.

In some instances, the disclosed methods for performing nucleic acid sequencing (e.g., in vitro and/or flow cell sequencing) may comprise performing one or more steps (e.g., 1, 2, 3, 4, 5, or more than 5) steps of nucleic acid amplification. Amplification reactions with respect to in situ based sequencing methods as described herein are discussed previously.

Nucleic acid amplification may be performed using any of a variety of nucleic acid amplification techniques known to those of skill in the art, including both thermal and/or isothermal nucleic acid amplification techniques. Examples of suitable thermal nucleic acid amplification techniques include, but are not limited to, polymerase chain reaction (PCR), multiplexed PCR, nested PCR, bridge PCR, reverse transcription PCR (RT-PCR). Examples of suitable isothermal nucleic acid amplification techniques include, but are not limited to, rolling circle amplification (RCA), nucleic acid sequence-based amplification (NASBA), loop-mediated isothermal amplification (LAMP), strand displacement amplification (SDA), helicase-dependent amplification (HDA), nicking enzyme amplification reaction (NEAR), and recombinase polymerase amplification (RPA). Examples of methods for performing nucleic acid amplification are described in, for example, Gill et al. (2008), “Nucleic Acid Isothermal Amplification Technologies—A Review”, Nucleosides, Nucleotides, and Nucleic Acids 27:224-243, Fakruddin et al. (2013), “Nucleic acid amplification: Alternative method of polymerase chain reaction”, J Pharm Bioallied Sci. 5 (4): 245-252, and U.S. Pat. No. 8,143,008, the entire contents of each of which are incorporated herein by reference. In some embodiments, the amplification reaction is a rolling circle amplification

In some instances, the disclosed methods for performing nucleic acid sequencing (e.g., in situ and/or flow cell sequencing) can comprise the use of primer sequences that are complementary to, e.g., a subsequence (or primer binding site) that is part of an endogenous nucleic acid target sequence or a sequence (or primer binding site) that is located at or near a barcode (identifier) sequence associated with a target analyte. In some instances, a primer sequence may be designed to hybridize to a primer binding site associated with a single target analyte sequence and/or an associated target-specific barcode sequence. In some instances, a primer sequence may be designed to hybridize to a sequence (or primer binding site) that is associated with a plurality of target analyte sequences and/or associated target-specific barcode sequences (e.g., at least 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000, or more than 1000 target analyte sequences and/or associated target-specific barcode sequences). In some instances, a primer sequence may be designed to hybridize to a probe sequence (e.g., a sequence present in a padlock probe). In some embodiments, a plurality of target sequences comprising a first subset of target sequences and a second subset of target sequences are sequenced simultaneously, and the first subset of target sequences are sequenced using a first primer sequence, and the second subset of target sequences are sequenced using a second primer sequence. Using multiple different primer sequences may be useful, for example, to reduce optical crowding.

In some instances, the disclosed methods for performing nucleic acid sequencing (e.g., in situ and/or flow cell sequencing) may comprise performing one or more steps of nucleic acid amplification or replication using one or more polymerases. Examples of polymerases that may be used for amplification include, but are not limited to, DNA polymerases (e.g., Taq DNA polymerase), RNA polymerases, and/or reverse transcriptases.

As noted elsewhere herein, non-limiting examples of polymerases for use in rolling circle amplification (RCA) comprise DNA polymerases such phi29 (29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some aspects, the polymerase is phi29 DNA polymerase.

As noted elsewhere herein, the disclosed methods for performing nucleic acid sequencing (e.g., in situ sequencing) may comprise inferring the sequence of a template nucleic acid molecule from a series of optical signals (e.g., fluorescence signals) detected in images acquired during a repetitive series of sequencing reaction cycles in a process referred to as “base-calling”. The interplay of sequencing chemistry, opto-fluidics hardware, optical sensors, and signal processing software utilized in sequencing platforms affects the types of errors made during sequencing (see, e.g., Lederberger et al. (2011), “Base-calling for next-generation sequencing platforms”, Brief Bioinform. 12(5): 489-497). The characterization of errors associated with the sequencing process and implementation of chemistry-, imaging-, and/or signal processing software-based methods for minimizing sequence errors are thus important for maximizing the accuracy of sequencing results.

In four-color sequencing methods, for example, a set of four images-one image for each of four detection channels corresponding to the emission wavelengths for four fluorophores used to label the reversibly terminated nucleotides—are acquired in each sequencing cycle. Processing of the images to detect fluorescence intensity signals produces an intensity quadruple for the location of each sequencing colony on a flow cell surface or the location of each target analyte, or amplified representation thereof (e.g., an RCP) in the case of in situ sequencing, where each value represents the intensity of the fluorescence signal for the detection channels corresponding to A, C, G and T. Ideally, the channel in which the maximum intensity occurs would be the base that is “called” for a given RCP or sequencing colony (or target analyte) in a given cycle. However, the chemical processes involved in sequencing are imperfect, leading to errors in base-calling (see, e.g., Cacho, et al. (2016), “A Comparison of Base-calling Algorithms for Illumina Sequencing Technology”, Briefings in Bioinformatics 17 (5): 786-795). In some sequencing-by-synthesis (SBS) platforms, for example, sources of error may include phasing (or lagging; e.g., where the primed template nucleic acid molecules at one or more locations fail to incorporate the next base due to variation in polymerase reaction kinetics), pre-phasing (or leading; e.g., where more than one nucleotide is incorporated in a single cycle due to, e.g., impurities in the reversibly terminated nucleotides), signal decay (due to, e.g., photobleaching and/or loss of template nucleic acid during the sequencing process), and cross-talk (e.g., when two or more fluorophore emission spectra overlap, which may cause a positive correlation between signal intensities measured in the corresponding detection channels).

A variety of statistical approaches have been developed to correct for, or minimize, such errors and generate more accurate base-calls. Examples include, but are not limited to, AYB (Goldman Group, European Molecular Biology Laboratory—European Bioinformatics Institute, Cambridgeshire, UK), and Bustard (Illumina, Inc., San Diego, CA).

The output of the base-calling process applied to optical signals detected in a series of images of a biological sample or flow cell surface acquired during a cycling sequencing process consists of a plurality of sequence reads, e.g., the nucleotide sequences determined for all or a portion of a template nucleic acid molecule (e.g., an endogenous nucleic acid analyte or a barcode sequence associated with a target analyte).

In some instances, the sequence reads generated using the disclosed sequencing methods may comprise sequence reads of at least about 5, 10, 20, 30, 40, 50, 60, 70, 80, 90, 100 or more nucleotides or base pairs of the template nucleic acid sequences.

In some instances, the disclosed sequencing methods may generate at least about 100, 200, 300, 400, 500, 600, 700, 800, 900, 1,000, or more sequencing reads per run. In some instances, the disclosed method may generate at least about 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, or more than 10⁶, sequencing reads per run.

In some instances, the disclosed sequencing methods include assembly of longer template nucleic acid sequences, e.g., genome fragments or whole genomes, from a plurality of relatively short sequence reads. Sequence assembly may be performed by identifying the overlapping sequences from multiple short sequence reads to assemble longer, contiguous sections of sequence.

In some instances, the disclosed sequencing methods include identifying a code word corresponding to a sequence read or an assembled sequence, where the code word is one of a plurality of code words in a codebook that includes assignment of each of the plurality of code words to a target analyte of interest. The sequence read or assembled sequence may thus be used to identify a specific target analyte (based on the corresponding code word) in, e.g., a multiplexed in situ detection or sequencing assay.

In some instances, the disclosed sequencing methods include alignment of sequence reads and/or assembled sequences to a known reference sequence or consensus sequence (e.g., the GRCh38 human reference genome (Genome Reference Consortium)) from the same or a similar organism. Alignment to a reference sequence or consensus sequence may be used to identify gaps, errors, or variants in the assembled sequence. Any of a variety of bioinformatics software programs known to those of skill in the art may be used to assemble longer sequences from relatively short sequence reads. Examples include, but are not limited to, DBG2OLC (see, e.g., Ye et al. (2016), “DBG2OLC: Efficient Assembly of Large Genomes Using Long Erroneous Reads of the Third Generation Sequencing Technologies”, Scientific Reports 6:31900), SPAdes (see, e.g., Bankevich et al. (2012), “SPAdes: A New Genome Assembly Algorithm and Its Applications to Single-Cell Sequencing”, J. Computational Biol. 19(5): 455-477), SparseAssembler (see, e.g., Ye et al. (2012), “Exploiting Sparseness in de novo Genome Assembly”, BMC Bioinformatics 13(Suppl 6):S1), Fermi (see, e.g., Li (2012), “Exploring Single-Sample SNP and INDEL Calling with Whole-Genome de novo Assembly”, Bioinformatics 28(14):1838-1844), and String Graph Assembler (SGA) (see, e.g., Simpson et al. (2012), “Efficient de novo Assembly of Large Genomes Using Compressed Data Structures”, Genome Res. 22:549-556).

In some instances, the sequencing methods described herein (e.g., in situ sequence sequencing) include using instruments having integrated optics and fluidics modules (“opto-fluidic instruments” or “opto-fluidic systems”) for detecting target molecules (e.g., nucleic acids, proteins, antibodies, etc.) in biological samples (e.g., one or more cells or a tissue sample) as described herein.

In an opto-fluidic instrument, the fluidics module is configured to deliver one or more reagents (e.g., P-modified nucleotide molecules, primers, detectable-labeled probes and/or non-labeled probes, polymerases and/or other enzymes, deprotection reagents, buffers, etc.) to the biological sample (e.g., to a sample cartridge within which the biological sample is contained) and/or to remove spent reagents therefrom. In some instances, one or more sample preparation steps (e.g., fixing, embedding, and/or sample clearing) may be performed prior to the sample being placed on the instrument. In some instances, the fluidics module is configured to deliver one or more further reagents (e.g., primary probe(s) such as circular probe(s) or circularizable probe(s) or probe set(s)) and/or to remove non-specifically hybridized probe(s). In some instances, the fluidics module is configured to deliver one or more detectably labeled probes and optionally intermediate probes to detect the target analytes, or amplified representatives thereof (e.g., RCP(s)) in the biological sample. In some instances, the fluidics module is configured to deliver one or more nucleotide mixtures (e.g., mixtures of P-modified nucleotide molecules, as well as primers, polymerases, deprotection reagents, etc.) to sequence, e.g., native nucleic acid sequences, barcode sequences associated with target analytes, or amplified copies thereof (e.g., barcode sequences included in RCP(s)) in the biological sample.

Additionally, the optics module is configured to illuminate the biological sample with light having one or more spectral emission curves (over a range of wavelengths) and subsequently capture one or more images of emitted light signals from the biological sample during one or more decoding (e.g., probing or sequencing) cycles. In various instances, the captured images may be processed in real time and/or at a later time to determine the presence of the one or more target molecules in the biological sample, as well as two-dimensional and/or three-dimensional position information associated with each detected target molecule within the biological sample. In various instances, the captured images of a flow cell surface may be processed in real time and/or at a later time to determine the sequence of the one or more nucleic acid sequences (e.g., barcode sequences associated with one or more target molecules) that have been extracted from a biological sample. In some embodiments, the optics module further comprises an autofocus mechanism configured to maintain focus at a specified sample plane (e.g., a plane that is perpendicular to the optical axis of an objective lens of the optics module).

Additionally, the opto-fluidics instrument includes a sample module configured to receive (and, optionally, secure) one or more biological samples (e.g., biological samples contained with one or more sample cartridges). In some instances, the sample module includes an X-Y stage configured to move the biological sample along an X-Y plane (e.g., perpendicular to the optical axis of an objective lens of the optics module).

In various instances, the opto-fluidic instrument is configured to analyze one or more target molecules (e.g., one or more target RNAs) in their naturally occurring place (i.e., in situ) within the biological sample. In some instances, the opto-fluidic instrument is configured to analyze one or more target RNAs in relative spatial locations within the biological sample. For example, an opto-fluidic instrument may be an in-situ analysis system used to analyze a biological sample and detect target molecules including, but not limited to, DNA, RNA, proteins, antibodies, and/or the like. In some instances, the in situ analysis system is used to detect one or more target RNAs using target-primed rolling circle amplification (RCA) according to the methods disclosed herein.

In various instances, the opto-fluidic instrument may be configured to perform in situ target molecule detection via base-by-base sequencing (e.g., by sequencing an identifier sequence such as a barcode sequence associated with a target molecule) and/or any imaging or target molecule detection technique. That is, for example, an opto-fluidic instrument may include a fluidics module that includes fluids needed for establishing the experimental conditions required for the probing or sequencing of target molecules (or associate barcode sequences) in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

In various instances, the sample analyzed is a biological sample (e.g., a tissue) that includes molecules such as DNA, RNA, proteins, antibodies, etc. For example, the sample can be a sectioned tissue that is treated to access the RNA thereof for probe (e.g., circularizable probe) hybridization and sequencing (e.g., using a sequencing primer that hybridizes to RCPs to sequence barcode sequences in the RCPs) described elsewhere herein.

In various instances, the sample is placed in the opto-fluidic instrument or system for analysis and detection of the molecules in the sample. In various instances, the opto-fluidic instrument or system is configured to facilitate the experimental conditions conducive for the detection of the target molecules. For example, the opto-fluidic instrument or system can include a fluidics module, an optics module, a sample module, and an ancillary module, and these modules may be operated by a system controller to create the experimental conditions for base-by-base sequencing of nucleic acid molecules in the sample, as well as to facilitate the imaging of the sample (e.g., by an imaging system of the optics module). In various instances, the various modules of the opto-fluidic instrument or system include components in communication with each other, or at least some of them may be integrated together.

In various instances, the sample module is configured to receive the sample into the opto-fluidic instrument or system. For instance, the sample module may include a sample interface module (SIM) that is configured to receive a sample device (e.g., cassette) onto which the sample can be deposited. That is, the sample may be placed in the opto-fluidic instrument or system by depositing the sample (e.g., the sectioned tissue) on a sample device that is then inserted into the SIM of the sample module. In some instances, the sample module may also include an X-Y stage onto which the SIM is mounted. The X-Y stage may be configured to move the SIM mounted thereon (e.g., and as such the sample device containing the sample inserted therein) in perpendicular directions along the two-dimensional (2D) plane of the opto-fluidic instrument or system.

The experimental conditions that are conducive for the detection of the molecules in the sample may depend on the target molecule detection technique that is employed by the opto-fluidic instrument or system. For example, in various instances, the opto-fluidic instrument or system can be a system that is configured to detect molecules (e.g., nucleotides incorporated into extending sequencing primers using an identifier sequence as a template) in the sample.

In various instances, the fluidics module may include one or more components that may be used for storing the reagents, as well as for transporting said reagents to and from the sample device containing the sample. For example, the fluidics module may include reservoirs configured to store the reagents, as well as a waste container configured for collecting the reagents (e.g., and other waste) after use by the opto-fluidic instrument or system to analyze and detect the molecules of the sample. Further, the fluidics module may also include pumps, tubes, pipettes, etc., that are configured to facilitate the transport of the reagent to the sample device (e.g., and as such the sample). For instance, the fluidics module may include pumps (“reagent pumps”) that are configured to pump washing/stripping reagents to the sample device for use in washing/stripping the sample (e.g., as well as other washing functions such as washing an objective lens of the imaging system of the optics module).

In various instances, the ancillary module can be a cooling system of the opto-fluidic instrument or system, and the cooling system may include a network of coolant-carrying tubes that are configured to transport coolants to various modules of the opto-fluidic instrument or system for regulating the temperatures thereof. In such cases, the fluidics module may include coolant reservoirs for storing the coolants and pumps (e.g., “coolant pumps”) for generating a pressure differential, thereby forcing the coolants to flow from the reservoirs to the various modules of the opto-fluidic instrument or system via the coolant-carrying tubes. In some instances, the fluidics module may include returning coolant reservoirs that may be configured to receive and store returning coolants, e.g., heated coolants flowing back into the returning coolant reservoirs after absorbing heat discharged by the various modules of the opto-fluidic instrument or system. In such cases, the fluidics module may also include cooling fans that are configured to force air (e.g., cool and/or ambient air) into the returning coolant reservoirs to cool the heated coolants stored therein. In some instances, the fluidics module may also include cooling fans that are configured to force air directly into a component of the opto-fluidic instrument or system so as to cool said component. For example, the fluidics module may include cooling fans that are configured to direct cool or ambient air into the system controller to cool the same.

As discussed above, the opto-fluidic instrument or system may include an optics module which includes the various optical components of the opto-fluidic instrument or system, such as but not limited to a camera, an illumination module (e.g., LEDs), an objective lens, and/or the like. The optics module may include a fluorescence imaging system that is configured to image the fluorescence emitted by the detectably labeled nucleotides are incorporated in extending sequencing primers in the sample after the detectable labels are excited by light from the illumination module of the optics module.

In some instances, the optics module may also include an optical frame onto which the camera, the illumination module, and/or the X-Y stage of the sample module may be mounted.

In various instances, the system controller may be configured to control the operations of the opto-fluidic instrument or system (e.g., and the operations of one or more modules thereof). In some instances, the system controller may take various forms, including a processor, a single computer (or computer system), or multiple computers in communication with each other. In various instances, the system controller may be communicatively coupled with data storage, set of input devices, display system, or a combination thereof. In some cases, some or all of these components may be considered to be part of or otherwise integrated with the system controller, may be separate components in communication with each other, or may be integrated together. In other examples, the system controller can be, or may be in communication with, a cloud computing platform.

In various instances, the opto-fluidic instrument or system may analyze the sample and may generate the output that includes indications of the presence of the target molecules in the sample. For instance, with respect to instances discussed above where the opto-fluidic instrument or system employs a sequencing technique for detecting molecules, the opto-fluidic instrument or system may cause the sample to undergo successive sequencing cycles, where during the same sequencing cycle the sample is imaged to detect signals associated with nucleotide binding and/or incorporation events at some locations in the sample, as well as to detect an absence of signals at other locations in the sample. In such cases, the output may include a series of optical signals (e.g., a code word) specific to each identifier sequence (e.g., a barcode sequence), which allow the identification of the target molecules.

### III. Compositions and Kits

In some aspects, provided herein are compositions comprising any of the P-modified nucleotides, primers, polymerases, and/or primary probes (e.g., circular probes or circularizable probes or probe sets) described herein.

In some instances, provided herein is a kit comprising any of the P-modified nucleotide molecules described herein. In some instances, the kit further comprises any of the primers described herein. In some instances, provided herein is a kit further comprising any of the polymerases described herein.

In some instances, provided herein is a kit for sequencing comprising a plurality of P-modified nucleotide molecules as described herein, and one or more additional reagents for performing the sequencing reaction. In some instances, the one or more additional reagents are selected from: a polymerase, a primer, modified 3′ reversibly terminated nucleotide molecules, a flow cell, primers, and adapters for sequencing library preparation, or any combination thereof. In some embodiments, the one or more additional reagents include a polymerase. In some embodiments, the polymerase is a thermostable polymerase. In some embodiments, the polymerase is a polymerase permissive for a 3′ blocking group. In some embodiments, the one or more additional reagents include a primer, such as a sequencing primer.

In some instances, provided herein is a kit for performing in situ sequencing comprising a plurality of P-modified nucleotide molecules as described herein, and one or more additional reagents for performing the in situ sequencing reaction. In some instances, the one or more additional reagents include a polymerase, a primer, modified, a support for a tissue or cell sample (e.g., a slide), or any combination thereof. In some instances, the kit further comprises any of the circular probes and/or circularizable probes or probe sets disclosed herein. In some instances, the kit includes a polymerase for rolling circle amplification, and optionally dNTPs for the rolling circle amplification.

The various components of the kit may be present in separate containers or certain compatible components may be pre-combined into a single container. In some instances, the kits further contain instructions for using the components of the kit to practice the provided methods. In some instances, sets of P-modified nucleotide molecules having each nucleobase type (as described elsewhere) may be provided together in a single container, such as a tube. In some instances, the P-modified nucleotide molecules of each nucleobase type may be provided in separate containers. In some instances, a first combination of P-modified nucleotide molecules comprising (e.g., two of four nucleobase types) are provided together in a first container, and a second combination of P-modified nucleotide molecules (e.g., of the other two of four nucleobase types) may be provided in a second container.

In some aspects, provided herein is a kit for sequencing a template nucleic acid molecule, comprising: a plurality of P-modified nucleotide molecules as described herein; a primer designed to hybridize to the template nucleic acid molecule; and a polymerase. In some instances, the plurality of P-modified nucleotide molecules comprises four sets of P-modified nucleotide molecules, wherein each of the four sets of P-modified nucleotide molecules comprises a different nucleobase and a different dye. In some instances, molecules of three of the four different P-modified nucleotide molecules are coupled to different dyes, and molecules of one of the four different nucleobase types are not conjugated to a fluorophore.

In some embodiments, the kits contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits optionally contain other components, for example nucleic acid primers.

### IV. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” and “nucleic acid molecule,” used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

In the present description, the term “about” means±20% of the indicated range, value, or structure, unless otherwise indicated. The term “consisting essentially of” limits the scope of a claim to the specified materials or steps and those that do not materially affect the basic and novel characteristics of the claimed subject matter. As used herein, the terms “include” and “have” are used synonymously, which terms and variants thereof are intended to be construed as non-limiting. The term “comprise” means the presence of the stated features, integers, steps, or components as referred to in the claims, but that it does not preclude the presence or addition of one or more other features, integers, steps, components, or groups thereof.

All publications, comprising patent documents, scientific articles and databases, referred to in this application are incorporated by reference in their entirety for all purposes to the same extent as if each individual publication were individually incorporated by reference.

### EXAMPLES

The following examples are included for illustrative purposes only and are not intended to limit the scope of the present disclosure.

### Example 1: Generating Peptide-Modified Nucleotide Molecules

This example provides methods for generating peptide-modified nucleotides having the structure N-L-P-D, wherein N is a nucleotide comprising a sugar and a base, L is a linker, P is a peptide, and D is a dye.

In a first reaction, 1 uL peptide having the sequence KDDDDDDC (SEQ ID NO: 1) in solution (25 mM in 50/50 DMSO/water), 25 uL a rhodamine-derived dye NHS ester (10 mM in DMSO), 14 uL nuclease-free water, and 10 uL NaHCO₃ (500 mM in water) were mixed, and shaken at room temperature overnight. The molar ratio between dye and peptide was 10:1.

In a second reaction, 100 uL peptide-dye (40 uM in water), 1 uL DTT (1 M in water), and 2 uL sodium phosphate (500 mM in water were mixed and incubated at 50 C for 1 hour. 20 uL 3 M sodium acetate (pH 5.5) and 1 mL acetone were added to the solution, and the solution was stored at −20 C for 1 hour. The solution was centrifuged at 12000 rpm for 1 hour, and the supernatant was discarded. The precipitate was washed twice with acetone, and re-suspended in 60 uL PBS.

In a third reaction, 60 uL peptide-dye (64 uM) and 9 uL a modified dTTP (in DMF) having the following structure:

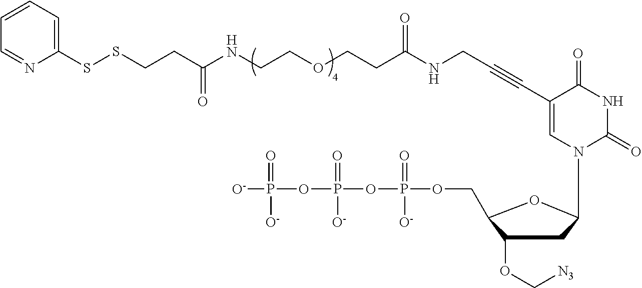

were mixed, and shaken at room temperature overnight. The molar ratio between peptide and HE0309 was 1:4. The product was purified by reverse-phase HPLC. The resulting peptide-modified nucleotide had the following structure:

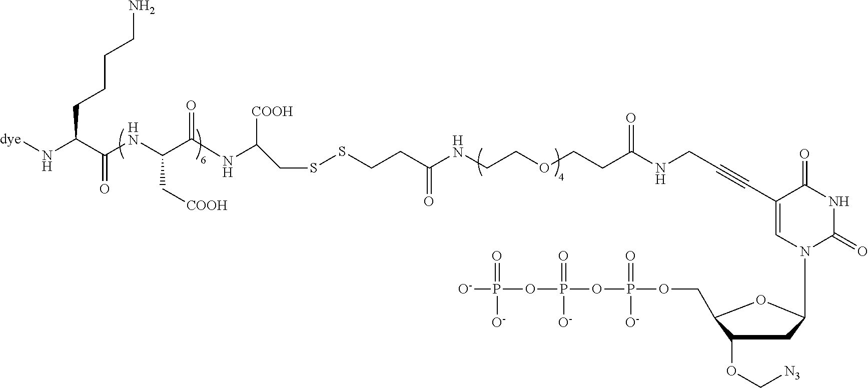

### Example 2: Nucleotide Incorporation In Vitro

This example demonstrates incorporation of a peptide-modified nucleotide molecule as described in Example 1 into a priming strand in an in vitro assay. The incorporation events were assayed and shown by gel electrophoresis.

Incorporation efficiency for the peptide-modified nucleotide molecule generated in Example 1 was assayed. Reagents as shown in Table 1 were used for the incorporation reaction.

TABLE 1

| Reagent | Final Conc. |
|---|---|
| Polymerase buffer | 1x |
| RT (uM) | 1.00 |
| Primer (uM) | 0.2 |
| Exo- family B | 3.00 |
| polymerase (uM) | |

For the assay, a template DNA strand was annealed to a primer conjugated to a dye, with the template strand having an ‘A’ at the position proximal to the 3′ end of the primer. Next, polymerase molecules and a plurality of peptide-modified 3′ blocked dTTP molecules were added under conditions allowing for primer extension to incorporate a single peptide-modified nucleotide molecule. The incorporation was performed at two different temperatures: 45 C and 60 C. The reaction products were separated on a 15% TBE Urea gel and imaged using a first channel to detect the dye conjugated to the primer, and a second channel to detect the dye conjugated to the peptide-modified nucleotide molecule or the control dTTP. Using the second channel, incorporation of the peptide-modified nucleotide molecule would be indicated by a band higher than the band of the unreacted peptide-modified nucleotide molecule, indicating a higher molecular weight. The gels were imaged for quantification, and percent incorporation was calculated. At 45 C, the incorporation efficiency for the peptide-modified nucleotide was 78.2, and at 60 C, the incorporation efficiency was 100%. Thus, the peptide-modified nucleotide molecule is incorporable into a priming strand.

### Example 3: P-Modified Nucleotide Incorporation into a Primed Rolling Circle Product (RCP) In a Tissue Sample In Situ

This example describes a protocol for use of a peptide-modified (P-modified) nucleotide molecule for an in situ sequencing reaction performed on a tissue sample. The P-modified nucleotide molecules of this example include molecules as generated in Example 1.

A cryosectioned tissue sample (fixed and permeabilized) is placed onto a glass slide for processing. Next, a circularizable probe is added to the slide for probe hybridization during an overnight incubation at room temperature. The probe targets a control mRNA known to be expressed in the tissue (e.g., GAPDH) and includes a sequencing primer binding site and a barcode sequence that is associated with the target sequence (e.g., codes for the target sequence). The circularizable probe is allowed to hybridize to the target sequence. The circularizable probe binds to two sequences separated by a gap, and after hybridization the gap is filled using a polymerase reaction. The tissue sample is then contacted with a ligation reaction mix including ligase for two hours at 37° C., and the circularizable probe is ligated to form a circular template for rolling circle amplification (RCA). A post-ligation wash is performed and the tissue sample is then incubated with an RCA mixture containing a DNA polymerase and dNTPs for RCA of the circularized probes, and amplified for 1 to 3 hours at a temperature of between 25° C. and 35° C. From this amplification, the RCA products (“RCPs”) are generating including multiple copies of the probe target binding regions, the primer binding site, and the barcode sequence.

Next, a sequencing reaction cycle is performed. The sequencing reaction uses a sequencing primer that binds to the primer binding site upstream of the barcode, a polymerase, and a composition including first P-modified nucleotides of a first nucleobase type and having a first dye, a second P-modified nucleotide of a second nucleobase type and having a second dye, a third P-modified nucleotide of a second nucleobase type and having a third dye, and a fourth P-nucleotide molecule of a fourth nucleobase type and having a fourth dye. Next, the reaction is incubated for at least 10 seconds, allowing for incorporation of a P-modified nucleotide into the priming strand, when the nucleobase type is complementary to the RCP nucleobase at the polymerase active site. A first image is taken in a channel for detecting the fourth dye type, to register the location of the RCP on the slide. Additional images are performed in channels for detecting the first, second, and third dyes. Detection of either the first dye, the second dye, the third dye, or the fourth dye is indicative of incorporation of the first nucleobase, second nucleobase, third nucleobase, or fourth nucleobase respectively. Next, the sample is washed, and then treated with a reducing agent to cleave the linker and remove the peptide and dye. The reducing agent also de-blocks the 3′ reversible blocker. The sequencing reaction is then repeated for a total of at least 4 cycles to obtain a sequence of the barcode.

What is claimed is:

1. A peptide-modified nucleotide having the structure: 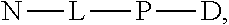
wherein N is a nucleotide comprising a 3′ blocked sugar and a base, L is a linker, P is a peptide comprising a charged residue, and D is a dye, and
wherein the linker is attached to the base of the nucleotide.

2. The peptide-modified nucleotide of claim 1, wherein the sugar comprises a ribose.

3. The peptide-modified nucleotide of claim 1, wherein the sugar comprises a 2′-deoxyribose.

4. The peptide-modified nucleotide of claim 1, wherein a 5′ phosphoryl group of the sugar comprises a monophosphate, diphosphate, or triphosphate, optionally wherein the 5′ phosphoryl group is a triphosphate.

5. The peptide-modified nucleotide of claim 1, wherein the 3′ blocked sugar comprises a 3′-OR, wherein R is selected from the group consisting of: azidomethyl, allyl, methyl, methyl carbamate, hydroxymethyl, amine, ester, disulfide, sulfate, and phosphate.

6. The peptide-modified nucleotide of claim 1, wherein the base is selected from the group consisting of: an adenine, an analogue of adenine, a cytosine, an analogue of cytosine, a guanine, an analogue of guanine, a thymine, an analogue of thymine, a uracil, and an analogue of uracil.

7. The peptide-modified nucleotide of claim 1, wherein the peptide is 4 to 30 amino acids in length.

8. The peptide-modified nucleotide of claim 1, wherein more than 50% of residues of the peptide are charged residues.

9. The peptide-modified nucleotide of claim 1, wherein the peptide comprises a negatively charged residue.

10. The peptide-modified nucleotide of claim 1, wherein the peptide comprises a cysteine residue or a cysteine derivative.

11. The peptide-modified nucleotide of claim 10, wherein a sulfur of the cysteine derivative is attached to the dye or the linker.

12. The peptide-modified nucleotide of claim 1, wherein the peptide comprises an amine attached to the dye or the linker.

13. The peptide-modified nucleotide of claim 12, wherein the peptide comprises an amine attached to the linker.

14. The peptide-modified nucleotide of claim 1, wherein the peptide comprises the sequence X1-(X2)ₙ-X3, wherein n is 3 to 50, X1 is any amino acid or derivative thereof and is attached to one of the dye or the linker, X2 is any amino acid, and X3 is lysine or a cysteine derivative attached to the other of the dye or the linker.

15. The peptide-modified nucleotide of claim 14, wherein X1 is a lysine and comprises an N-terminally blocked amino group.

16. The peptide-modified nucleotide of claim 1, wherein the peptide has the structure X1-(X2)ₙ-X3, wherein:
n is 3 to 50,
X1 or X3 is a cysteine derivative comprising a sulfur attached to the linker, and:
(a) X1 is the cysteine derivative, X3 or an instance of X2 is a lysine having an amino side chain attached to the dye moiety, or
(b) when X3 is the cysteine derivative, either: (i) X1 is any amino acid having an N-terminus attached to the dye moiety, or (ii) X1 or an instance of X2 is a lysine having an amino acid chain attached to the dye moiety.

17. The peptide-modified nucleotide of claim 16, wherein X3 is the cysteine derivative, and X1 or an instance of X2 is the lysine having an amino acid chain attached to the dye moiety.

18. The peptide-modified nucleotide of claim 17, wherein X1 comprises a blocked N-terminal amino group.

19. The peptide-modified nucleotide of claim 1, wherein the peptide comprises an enzymatically cleavable sequence.

20. A composition comprising a plurality of peptide-modified nucleotides, comprising: a first peptide-modified nucleotide molecule having a first base type and a second peptide-modified nucleotide molecule having a second base type, wherein the first and the second peptide-modified nucleotide molecules each have the structure N-L-P-D, wherein: N is a nucleotide molecule comprising a 3′ blocked sugar and a base, L is a linker, P is a peptide, and D is a dye.

21. The composition of claim 20, wherein the dye of the first peptide-modified nucleotide molecules is a first dye, and the dye of the second peptide-modified nucleotide molecules is a second dye that differs from the first dye.

22. A method for sequencing a template nucleic acid molecule comprising:
(a) contacting a priming strand bound to a template nucleic acid molecule with a polymerase and a first plurality of nucleotide molecules comprising a first peptide-modified nucleotide having the structure N-L-P-D, wherein: N is a nucleotide molecule comprising a 3′ blocked sugar and a base, L is a linker, P is a peptide, and D is a dye, thereby incorporating the peptide-modified nucleotide into a 3′ terminus of the priming strand; and
(b) detecting a presence of the first peptide-modified nucleotide in the priming strand to identify a complementary nucleotide in the template nucleic acid molecule.

23. The method of claim 22, wherein the method further comprises, after the detecting, removing the 3′ blocking group of the first peptide-modified nucleotide from the priming strand.

24. A kit for sequencing a template nucleic acid molecule comprising: a plurality of peptide-modified nucleotide molecule having the structure N-L-P-D, wherein: N is a nucleotide molecule comprising a 3′ blocked sugar and a base, L is a linker, P is a peptide, and D is a dye; and a polymerase.


SEQUENCE LISTING (4 sequences)

SEQ ID NO: 1 - protein, 8 aa, synthetic construct
KDDDDDDC

SEQ ID NO: 2 - protein, 6 aa, synthetic construct
LVPRGS

SEQ ID NO: 3 - protein, 7 aa, synthetic construct
ENLYFQG

SEQ ID NO: 4 - protein, 6 aa, synthetic construct
  VARIANT 6: note = X = any amino acid
DDDDKX